(19) United States
(12) Patent Application Publication
(10) Pub. No.: US 2025/0137035 A1
(43) Pub. Date: May 1, 2025

(54) HYBRIDIZATION PROBES CONTAINING FLUORINATED CARBON CHAINS AND RELATED METHODS

(71) Applicants: ILLUMINA, INC., San Diego, CA (US); University of Utah Research Foundation, Salt Lake City, UT (US)

(72) Inventors: Alexander A. Gall, Kirkland, WA (US); Trevor F. Stockdale, Walnut Creek, CA (US); Brett Nels Anderson, Seattle, WA (US); Diana Bernat, Bothell, WA (US); Heng Xie, San Diego, CA (US); Robert Schlaberg, Salt Lake City, UT (US); Guochun Liao, Belmont, CA (US)

(21) Appl. No.: 18/687,723

(22) PCT Filed: Sep. 2, 2022
(86) PCT No.: PCT/US2022/075909

§ 371 Date: Feb. 28, 2024

Related U.S. Application Data
(60) Provisional application No. 63/240,642, filed on Sep. 3, 2021.

(51) Int. Cl. C12Q 1/6813

(52) U.S. Cl. CPC C12Q 1/6813

(57) ABSTRACT

In one aspect, disclosed herein are new hybridization probes that contain fluorinated carbon tags (F), methods of making these hybridization probes, and methods for using these hybridization probes for affinity capture of the probes both in purification during production and in the enrichment process using fluorous substrates. In certain embodiments, the hybridization probe comprises a) a polynucleotide having a 3′ end and a 5′ end and comprising about 20 to about 200 nucleotide units and b) one or more fluorinated affinity tags, wherein each affinity tag comprises one or more polyfluorinated carbon chains each comprising 3-30 carbon atoms; wherein the polynucleotide comprises a sequence complementary or substantially complementary to a target sequence within a target nucleic acid.

### CROSS-REFERENCE TO RELATED APPLICATIONS

This application claims priority to U.S. Provisional Application No. 63/240,642, filed Sep. 3, 2021, the content of which is incorporated by reference in its entirety.

### SEQUENCE LISTING

The present application is being filed along with a Sequence Listing in electronic format. The Sequence Listing is provided as a file entitled ILLINC.751.xml, created and last saved on Aug. 31, 2022, which is 27,443 bytes in size. The information in the electronic format of the Sequence Listing is hereby incorporated by reference in its entirety.

### TECHNICAL FIELD

The present disclosure is directed to nucleic acid hybridization probes that contain fluorinated carbon chains to facilitate their purification using fluorous substrates during production and to facilitate the isolation and enrichment of their complexes with target nucleic acids from complex nucleic acid samples.

### BACKGROUND

Next Generation Sequencing (NGS) is a powerful tool in identification of genetic markers of organisms including pathogens. As the costs of NGS continue to fall, metagenomics presents growing attraction as a method of identification of microorganisms in complex samples including clinical samples from patients. Such clinical samples after building libraries for sequencing usually represent primarily host DNA and RNA, while interest is focused on nucleic acids that belong to pathogens. Increase in selectivity and sensitivity of NGS in metagenomics can be achieved by a combination of bioinformatics and hybridization probe-based enrichment approaches. For example, Explify® software platform developed by IDbyDNA leverages ultra-rapid DNA search technology, AI-powered data interpretation, curated collections of millions of DNA sequences, comprehensive genotype-phenotype databases for AMR prediction, and user-friendly software interfaces to put Precision Metagenomics at the fingertips of laboratory personnel. Hybridization probe enrichment is synergistic with the software approach, and it allows to increase the relative presence of expected DNA targets in the pre-NGS libraries by hundreds and thousands of times. Most enrichment methods heavily rely on biotinylated hybridization probes based on DNA or RNA for capturing of hybrids by streptavidin beads. One of such enrichment approaches is described in Illumina patent application WO2020036991, COMPOSITIONS AND METHODS FOR IMPROVING LIBRARY ENRICHMENT. Massive parallel purification of such probes after automated oligo synthesis presents a big challenge. Illumina also published a method of making such hybridization probes using affinity purification (Highly parallel oligonucleotide purification and functionalization using reversible chemistry. Kerri T. York, Ryan C. Smith, Rob Yang, Peter C. Melnyk, Melissa M. Wiley, Casey M. Turk, Mostafa Ronaghi, Kevin L. Gunderson, Frank J. Steemers, Nucleic Acids Res. 2012 January; 40(1): e4. Published online 2011 Oct. 29).

Despite the advances in the development of nucleic acid hybridization probes and method for their use to isolate and enrich targeted nucleic acids noted above, a need exists for new nucleic acid hybridization probes that facilitate their purification during production and the isolation and enrichment of their complexes with target nucleic acids from complex nucleic acid samples. The present disclosure seeks to fulfill this need and provides further related advantages.

### SUMMARY

Disclosed herein are new hybridization probes that contain fluorinated carbon tags (FT), methods of making these hybridization probes, and methods for using these hybridization probes for affinity capture of the probes both in purification during production and in the enrichment process using fluorous substrates.

In one aspect, hybridization probes are disclosed.

In certain embodiments, the hybridization probe comprises a) a polynucleotide having a 3′ end and a 5′ end and comprising about 20 to about 200 nucleotide units and b) one or more fluorinated affinity tags, wherein each affinity tag comprises one or more polyfluorinated carbon chains each comprising 3-30 carbon atoms; wherein the polynucleotide comprises a sequence complementary or substantially complementary to a target sequence within a target nucleic acid.

As used herein the term “substantially complementary” refers to a sequence capable of hybridizing with the target sequence but that contains one or more mismatches.

In certain embodiments, the hybridization probe has the structure;

[(FT)ₙ-Y-L]ₘ-HyS

wherein:
FT is a fluorinated carbon affinity tag comprising one or more polyfluorinated carbon chains each comprising 3-30 carbon atoms;
n=is 1, 2, or 3;
m=1 or 2;
HyS is a polynucleotide having a 3′ end and a 5′ end and comprising about 20 to about 200 nucleotide units;
L is an optional linker moiety connecting HyS and Y, wherein L is an optionally substituted C2-C20 alkylene, or an optionally substituted C3-C20 heteroalkylene comprising 1-6 heteroatoms selected from P, O, N, S, and combinations thereof, wherein L can be optionally substituted with a duplex-stabilizing moiety selected from an intercalator and a minor groove binder (MGB); and
Y is an optionally substituted C2-C20 alkylene, optionally substituted C6-C10 arylene, optionally substituted C5-C10 heteroarylene, or an optionally substituted C3-C20 heteroalkylene comprising 1-6 heteroatoms selected from P, O, N, S, and combinations thereof.

In certain embodiments, L is a linear linker and may optionally include a stabilizer. In certain embodiments, Y is a doubler (2 FTs) or trebler (3FTs). In certain embodiments, L can be absent and then Y is directly attached to the HyS (no stabilizer). In certain embodiments, the one or more fluorinated affinity tags is attached to the 3′ end or at the 5′ end of the polynucleotide.

In other embodiments, the one or more fluorinated affinity tags is attached to the one or more nucleotide units.

In certain embodiments, the hybridization probe comprises two, three, four, or five fluorinated affinity tags.

In certain embodiments, at least one fluorinated affinity tag comprises two or more polyfluorinated carbon chains.

In certain embodiments of the hybridization probe, (FT)n-Y has two affinity tags and a structure defined by formula:

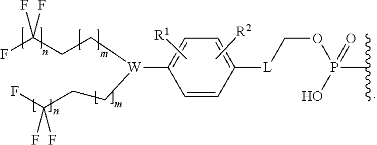

wherein:
each n, independently, is an integer from 5 to 18;
each m, independently, is 1 or 2;
W is N or a linking group comprising 1-20 carbon atoms and optionally 1-6 heteroatoms independently selected from P, O, N, and S;
R¹ and R² are independently selected from C1-C6 alkyl, halogen, nitro, amino, or cyano; or R¹ and R², together with the carbon atoms to which they are attached, can form a 5-7 membered ring optionally comprising 1-3 heteroatoms selected from P, O, N, and S; and
L is an optionally substituted C2-C20 alkylene, or an optionally substituted C3-C20 heteroalkylene comprising 1-6 heteroatoms selected from P, O, N, S, and combinations thereof.

In other embodiments of the hybridization probe, (FT)ₙ-Y has two affinity tags and a structure defined by formula:

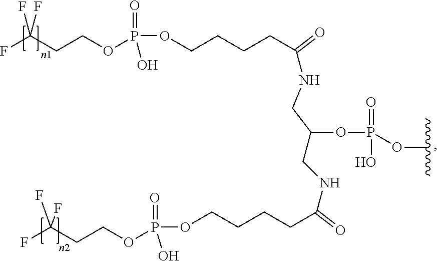

wherein n1 is an integer from 1 to 28; n2 is an integer from 1 to 28.

In further embodiments of the hybridization probe, (FT)ₙ-Y has three affinity tags and a structure defined by formula:

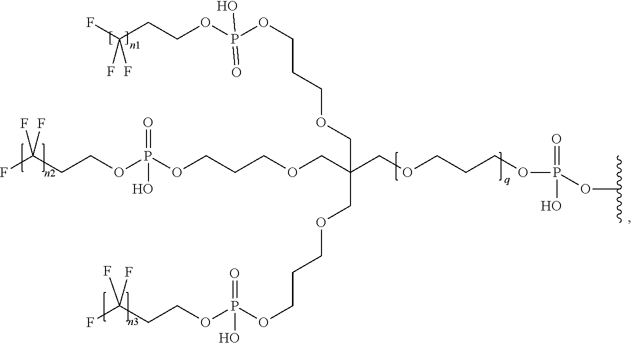

wherein n1 is an integer from 1 to 28; n2 is an integer from 1 to 28; n3 is an integer from 1 to 28; q is an integer from 0 to 10.

In certain embodiments of the hybridization probe, (FT)ₙ-Y has three affinity tags and a structure defined by formula:

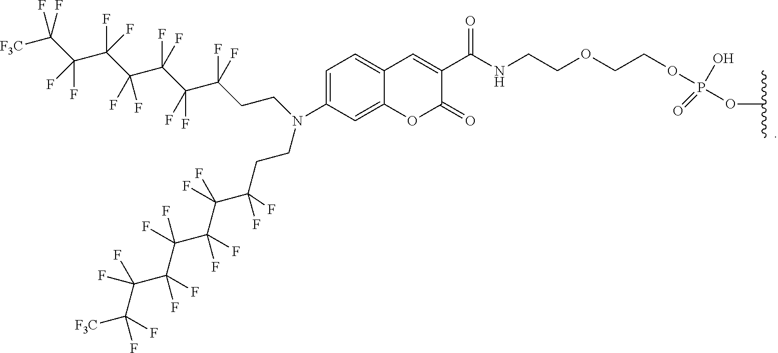 [SMILES: *OP(=O)(O)OCCOCCNC(=O)c1cc2ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc2oc1=O]

In other embodiments of the hybridization probe, (FT)ₙ-Y has three affinity tags and a structure defined by formula:

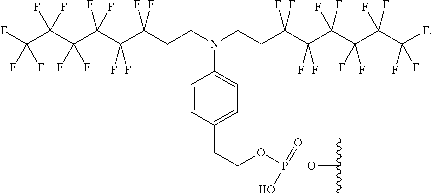 [SMILES: *OP(=O)(O)OCCc1ccc(N(CCC(F)(F)C(F)(F)C(F)(F)C(F)(F)C(F)(F)C(F)(F)F)CCC(F)(F)C(F)(F)C(F)(F)C(F)(F)C(F)(F)C(F)(F)F)cc1]

The hybridization probes described herein may include [(FT)ₙ-Y-L]ₘ- at the 5′, 3′, or any internal position of HyS.

In certain embodiments of the hybridization probe, the hybridization probe further comprises a stabilizing base, an intercalator, a minor groove binder, a biotin, a fluorescent dye, and/or a combination thereof. Minor groove binding agents non-covalently bind into the minor groove of double stranded DNA. Barton et al. define intercalators as “small organic molecules or metal complexes that unwind DNA in order to n-stack between two base pairs” (see Metallo-intercalators and metallo-insertors. Zeglis B M, Pierre V C, Barton J K, Chem Commun (Camb). 2007 Nov. 28; (44):4565-79.)

In certain embodiments of the method, the target nucleic acid is a microorganism nucleic acid or human nucleic acid.

In another aspect, the disclosure provides a composition comprising a plurality of hybridization probes as described herein, wherein the target nucleic acid is a microorganism nucleic acid and/or a human nucleic acid. In the practice of the invention, the target nucleic acid may be from any targeted species (animal, plant, etc.)

In a further aspect, the disclosure provides a method for enriching target nucleic acids in a mixed population of nucleic acids, wherein the mixed population of nucleic acids optionally comprises one or more target nucleic acids comprising a target sequence and one or more non-target nucleic acids, the method comprising the steps of:
a) contacting a first mixed population of nucleic acids with one or more hybridization probes as described herein, wherein the contacting is done under conditions sufficient for formation of a duplex between the one or more hybridization probes and the target sequence, thereby providing a second mixed population of nucleic acids wherein when the mixed population comprises one or more target nucleic acids, at least a portion of the target nucleic acids comprises duplexes with the one or more hybridization probes;
b) contacting the second mixed population of nucleic acids with an affinity substrate for a time sufficient to form a complex between the fluorinated affinity tag and the affinity substrate thereby binding at least a portion of the duplexes to the affinity support;
c) separating the unbound nucleic acids from the affinity support; and
d) dissociating the duplexes between the target nucleic acids and the one or more hybridization probes bound to the affinity support, thereby generating a third mixed population of nucleic acids, wherein the ratio of the target nucleic acids to non-target nucleic acids in the third mixed population is greater than the ratio of the target nucleic acids to non-target nucleic acids in the first mixed population.

It will be appreciated that in certain instances, the mixed population of nucleic acids does not include one or more target nucleic acids comprising a target sequence. In these instances, no hybridization is observed. An observation of no hybridization has diagnostic value.

In certain embodiments, the one or more target nucleic acids comprises viral nucleic acids, fungal nucleic acids, bacterial nucleic acids, parasite nucleic acids, drug resistance and/or pathogenicity markers, select host nucleic acids, parasitic nucleic acids, or nucleic acids from one or more anti-microbial resistance allele regions and/or combinations thereof.

In certain embodiments, the one or more target nucleic acids comprises human, animal, or plant nucleic acids.

In another aspect, the disclosure provides a method for enriching nucleic acids in a mixed population of nucleic acids, the method comprising the steps of:
a) contacting a first mixed population of nucleic acids with one or more first hybridization probes and one or more second hybridization probes, wherein the first mixed population of nucleic acids comprises one or more first target nucleic acids comprising a first target sequence and one or more second target nucleic acids comprising a second target sequence, wherein the one or more first hybridization probes comprises a first affinity tag and sequence complementary to the first target sequence, and wherein the one or more second hybridization probes comprises a second affinity tag and a sequence complementary to the second target sequence, and wherein the contacting is done under conditions sufficient for formation of a duplex between the one or more first hybridization probes and the first target sequence and/or between the one or more second hybridization probes and the second target sequence;
b) contacting the mixed population of nucleic acids of step a) with a second affinity support having affinity to the second affinity tag under the conditions sufficient for formation of a complex between the second affinity tag and the second affinity support, thereby binding at least a portion of the second nucleic acids to the second affinity support;
c) separating the unbound nucleic acids of step b) from the second affinity support;
d) contacting the unbound nucleic acids of step c) with a first affinity support having affinity to the first affinity tag under the conditions sufficient for formation of a complex between the first affinity tag and the first affinity support, thereby binding at least a portion of the first nucleic acids to the first affinity support;
e) separating the unbound nucleic acids of step d) from the first affinity support; and
f) dissociating the duplexes between the first target nucleic acids and the one or more first hybridization probes, thereby generating a second mixed population of nucleic acids, wherein the ratio of the first target nucleic acids to the second nucleic acids in the second mixed population is greater than the ratio of the first target nucleic acids to the second nucleic acids in the first mixed population.

The method described above uses two orthogonal affinity tags in the same hybridization mixture followed by selective separation.

As noted above, it will be appreciated that in certain instances, the mixed population of nucleic acids does not include one or more first target nucleic acids that comprise a target sequence and/or one or more second target nucleic acids that comprise a target sequence. In certain of these instances, no hybridization is observed. As also noted above, an observation of no hybridization has diagnostic value.

In certain embodiments, the one or more first target nucleic acids comprise viral nucleic acids, fungal nucleic acids, bacterial nucleic acids, parasite nucleic acids, drug resistance and/or pathogenicity markers, select host nucleic acids, parasitic nucleic acids, or nucleic acids from one or more anti-microbial resistance allele regions and/or combinations thereof.

In certain embodiments, the first hybridization probes are probes as described herein, the first affinity support is polyfluorinated polymer, the second affinity tag is biotin, and the second affinity support comprises avidin or streptavidin.

In certain embodiments, the second hybridization probes are probes as described herein, the second affinity support is polyfluorinated polymer, the first affinity tag is biotin, and the first affinity support comprises avidin or streptavidin.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a general structure of a representative hybridization probe (HyP) in accordance with the invention having one fluorinated tag (FT). HyP includes a Hybridizing Sequence (HyS) and an FT ligand that permits subsequent capture of the probe by fluorous surface or liquid.

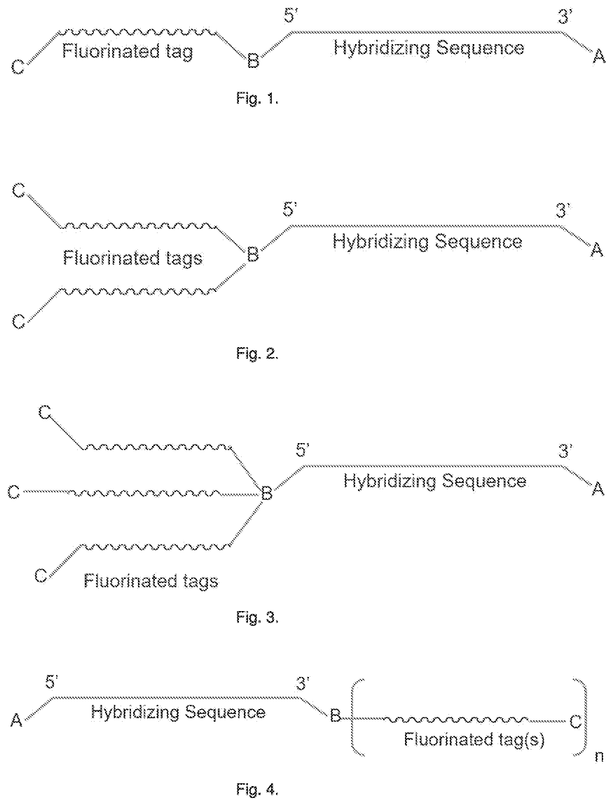

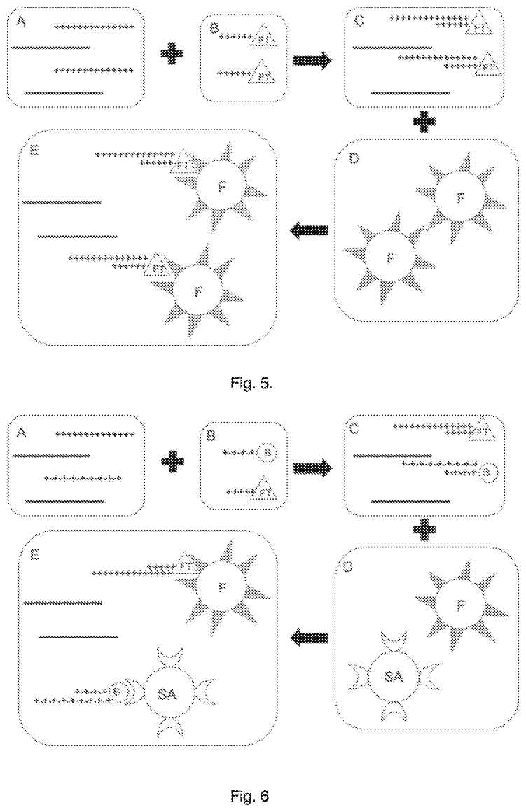

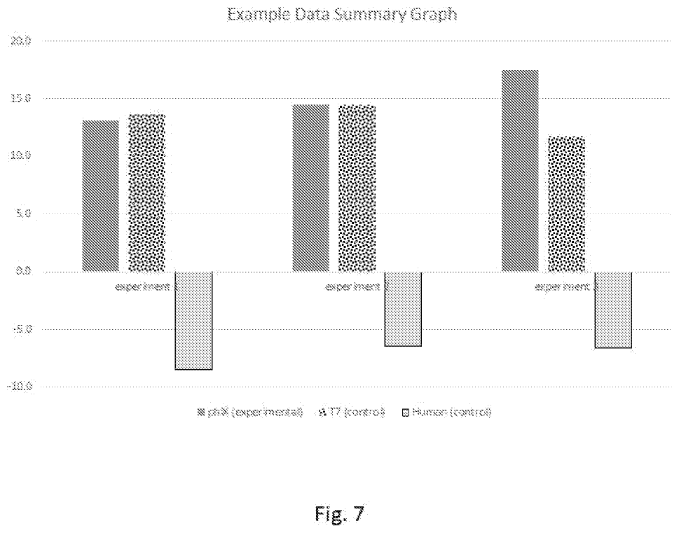

A is a 3′-terminating group of HyP that is connected to the 3′-position of terminal nucleotide of HyS through phosphate group to 3′-O group or directly to 3′-O of the terminal nucleoside base. A is H, alkyl, hydroxyalkyl, fluorinated alkyl, intercalating molecule, MGB, dye, biotin or reactive group selected from azido, terminal alkyne, NH₂, ketone, or aldehyde.

B is a linker that connected to the terminal nucleotide of HyS through phosphate group to 5′-O group or directly to 5′-O of the terminal nucleoside base. The linker is composed of alkyl, hydroxyalkyl, fluorinated alkyl, intercalating molecule, MGB, dye, biotin or reactive group selected from azido, terminal alkyne, NH₂, ketone, or aldehyde.

FT is an alkyl or oxyalkyl chain that consists of 5-20 carbon atoms with at least 5 carbon atoms represented as CF₂. The FT can be linear or branched or optionally oxyalkyl with 1-3 carbon atom(s) in the chain replaced with oxygen atom(s).

C is an optional terminal group connected to the FT directly or through phosphate group or oxygen atom and is composed of alkyl, hydroxyalkyl, fluorinated alkyl, intercalating molecule, MGB, dye, biotin or reactive group selected from azido, terminal alkyne, NH₂, ketone, or aldehyde.

FIG. 2 is a general structure of a representative hybridization probe (HyP) in accordance with the invention that contains two FTs. HyP includes a HyS and an FT ligand that permits subsequent capture of the probe by fluorous surface or liquid.

A is a 3′-terminating group of HyP that is connected to the 3′-position of terminal nucleotide of HyS through phosphate group to 3′-O group or directly to 3′-O of the terminal nucleoside base. A is H, alkyl, hydroxyalkyl, fluorinated alkyl, intercalating molecule, MGB, dye, biotin or reactive group selected from azido, terminal alkyne, NH₂, ketone, or aldehyde.

B is a linker that connected to the terminal nucleotide of HyS through phosphate group to 5′-O group or directly to 5′-O of the terminal nucleoside base. The linker is composed of alkyl, hydroxyalkyl, fluorinated alkyl, intercalating molecule, MGB, dye, biotin or reactive group selected from azido, terminal alkyne, NH₂, ketone, or aldehyde.

FT is an alkyl or oxyalkyl chain that consists of 5-20 carbon atoms with at least 5 carbon atoms represented as CF₂. The FT can be linear or branched or optionally oxyalkyl with 1-3 carbon atom(s) in the chain replaced with oxygen atom(s).

C is an optional terminal group connected to the FT directly or through phosphate group or oxygen atom and is composed of alkyl, hydroxyalkyl, fluorinated alkyl, intercalating molecule, MGB, dye, biotin or reactive group selected from azido, terminal alkyne, NH₂, ketone, or aldehyde.

FIG. 3 is a general structure of a representative hybridization probe (HyP) in accordance with the invention that contains three FTs. HyP includes a HyS and an FT ligand that permits subsequent capture of the probe by fluorous surface or liquid.

A is a 3′-terminating group of HyP that is connected to the 3′-position of terminal nucleotide of HyS through phosphate group to 3′-O group or directly to 3′-O of the terminal nucleoside base. A is H, alkyl, hydroxyalkyl, fluorinated alkyl, intercalating molecule, MGB, dye, biotin or reactive group selected from azido, terminal alkyne, NH-2, ketone, or aldehyde.

B is a linker that connected to the terminal nucleotide of HyS through phosphate group to 5′-O group or directly to 5′-O of the terminal nucleoside base. The linker is composed of alkyl, hydroxyalkyl, fluorinated alkyl, intercalating molecule, MGB, dye, biotin or reactive group selected from azido, terminal alkyne, NH₂, ketone, or aldehyde.

FT is an alkyl or oxyalkyl chain that consists of 5-20 carbon atoms with at least 5 carbon atoms represented as CF₂. The FT can be linear or branched or optionally oxyalkyl with 1-3 carbon atom(s) in the chain replaced with oxygen atom(s).

C is an optional terminal group connected to the FT directly or through phosphate group or oxygen atom and is composed of alkyl, hydroxyalkyl, fluorinated alkyl, intercalating molecule, MGB, dye, biotin or reactive group selected from azido, terminal alkyne, NH₂, ketone, or aldehyde.

FIG. 4 is a general structure of a representative hybridization probe (HyP) in accordance with the invention that contains one, two, or three (n=1, 2, or 3) FTs on the 3′-end of oligo. HyP includes a HyS and an FT ligand that permits subsequent capture of the probe by fluorous surface or liquid.

A is a 5′-terminating group of HyP that is connected to the 5′-position of terminal nucleotide of HyS through phosphate group to 5′-O group or directly to 5′-O or the terminal nucleoside base. A is H, alkyl, hydroxyalkyl, fluorinated alkyl, intercalating molecule, MGB, dye, biotin or reactive group selected from azido, terminal alkyne, NH₂, ketone, or aldehyde.

B is a linker that connected to the terminal nucleotide of HyS through phosphate group to 3′-O group or directly to 3′-O of the terminal nucleoside base. The linker is composed of alkyl, hydroxyalkyl, fluorinated alkyl, intercalating molecule, MGB, dye, biotin or reactive group selected from azido, terminal alkyne, NH₂, ketone, or aldehyde.

FT is an alkyl or oxyalkyl chain that consists of 5-20 carbon atoms with at least 5 carbon atoms represented as CF₂. The FT can be linear or branched or optionally oxyalkyl with 1-3 carbon atom(s) in the chain replaced with oxygen atom(s).

C is an optional terminal group connected to the FT directly or through phosphate group or oxygen atom and is composed of alkyl, hydroxyalkyl, fluorinated alkyl, intercalating molecule, MGB, dye, biotin or reactive group selected from azido, terminal alkyne, NH₂, ketone, or aldehyde.

FIG. 5 depicts the steps of a representative method using FT for enrichment of the targeted nucleic acids in accordance with the invention: (A) mixture of targeted (+++++) and untargeted (−−−−−−−) nucleic acids; (B) library of HyP labeled with FT; (C) HyP labeled with FT that are hybridized to targeted nucleic acids in a mixture with untargeted nucleic acids; (D) adding fluorous support to the mixture; and (E) pulling HyP hybrids with the targeted nucleic acid from the mixture by adsorbing on fluorinated support, thereby achieving separation from untargeted nucleic acids.

FIG. 6 depicts the steps of a representative method using orthogonal FT and biotin tags for enrichment of two sets of targeted nucleic acids in accordance with the invention: (A) mixture of the first targeted (+−+−+−), the second targeted (+++++) and untargeted (−−−−−−−) nucleic acids; (B) a mixture of two libraries of HyP, where one is labeled with biotin (B) and targeting the first set of targeted nucleic acids, and HyP labeled with FT and targeting the second set of targeted nucleic acids; (C) HyP labeled with biotin (B) and FT that are hybridized to corresponding two sets of targeted nucleic acids in a mixture with untargeted nucleic acids; (D) adding Streptavidin (SA) and fluorous (F) supports to the mixture; and (E) pulling HyP hybrids with the targeted nucleic acid from the mixture by adsorbing one set of targeted nucleic acids on fluorinated (F) and the second set on the Streptavidin (SA) supports, thereby achieving separation of the sets from each other and from untargeted nucleic acids.

FIG. 7 shows data for sample enrichment or depletion levels in certain experiments.

### DETAILED DESCRIPTION

A well-established conventional enrichment strategy uses hybridization probes tagged with biotin to hybridize to targeted DNA sequences followed by extraction using streptavidin-coated magnetic beads. A most common method of hybrid capture includes contacting the library with a probe wherein the probe hybridizes to a region of interest within a library member. The region of interest is separate from the adaptor region and includes genomic material of interest. The probe includes a biotin ligand that allows for subsequent capture of the probe with streptavidin surface.

Another method of capturing oligonucleotides is known in the art that includes tagging of oligos with fluorinated carbon chains that make such Fluorinated Tags (FT) to provide affinity to PTFE and other fluorinated carbon materials. Such FT labeling provided an advanced method for purification of oligos after automated synthesis (see, for example, W. H. Pearson, et al., Fluorous Affinity Purification of Oligonucleotides. J. Org. Chem. 2005, 70, 7114-7122). It has been shown that FT-tagged oligonucleotide probes that adsorbed to fluorinated surfaces can hybridize to complementary nucleic acids. For example, such oligos were adsorbed on fluorous-patterned surface and provided sequence-specific hybridization (Gabriella E. Flynn, et al., Reversible DNA micro-patterning using the fluorous effect. Chem. Commun., 2017, 53, 3094-3097). Another publication demonstrates the target DNA directs placement of FT-DNA molecules on the surface of DNA-gold nanoparticles, through sandwich hybridization, leading to the fluorous-tag driven generation of gold nanoparticles polymeric networks that enables the visual detection of target DNA either directly in aqueous solution or on a fluorinated substrate surface (Min Hong, et al., Nanoparticle-Based, Fluorous-Tag-Driven DNA Detection. Angew. Chem. 2009, 121, 9667-9670). However, FT have not been used for enrichment of the targeted nucleic acids in the presence of abundance of untargeted nucleic acids.

One embodiment relates to the discovery that fluorous affinity can be used in place of the streptavidin-biotin bond with the same hybridization targets. An advantage of leveraging fluorous affinity is the highly specific nature of fluorous-fluorous affinity, such that fluorinated solid sorbents and liquids with intrinsically low affinity for nucleic acids can be used as mediums of separation directly, which can enable higher levels of enrichment by lowering untargeted background DNA being carried into the final enriched sample.

This disclosure describes methods of hybrid capture for improving the efficiency of nucleic acid selection prior to sequencing including methods and compositions with a blocker and/or a hybridization buffer, and methods of using those in metagenomics applications. Specifically, the methods disclosed in the current invention utilize affinity of FT-tagged hybridization probes to fluorous materials such as surfaces and liquids that enable separation from nonhybridized nucleic acids. Additionally, PTFE and other fluorous surfaces are known to minimize binding to any molecules other than of a similar nature: fluorinated carbons. Such selectivity enhances separation of FT-labeled probes and their hybridized complexes from any untagged DNA, RNA, or proteins and common PCR inhibitors.

The following abbreviations are used throughout the disclosure:
FT Fluorinated carbon affinity Tag
HyS Hybridizing Sequence
HyP Hybridization Probe
MGB Minor Groove Binder
CPG Controlled Pore Glass
RM Reaction Mixture

In one aspect, the disclosure describes a nucleic acid hybridization probe (HyP) having the formula:

[(FT)ₙ-Y-L]ₘ-HyS

where (FT)ₙ is a fluorinated carbon affinity tag, comprises one or more polyfluorinated carbon chains each comprising 3-30 carbon atoms; n=1-3; m=1, 2; HyS is oligonucleotide having hybridization sequence with targeted nucleic acid. L is a linker connecting HyS and Y moieties and having 2-20 carbon atoms in the chain, some of which are optionally substituted with P, O, N and S atoms and may contain a duplex-stabilizing moiety such as intercalator or MGB. Y is another linker having 2-20 carbon atoms in the chain, some of which are optionally substituted with P, O, N and S atoms, connected to L and to one, two or three FT moieties that can be same or different.

In some embodiments, HyP is designed with at least one FT attached to 5′-end of HyS.

In some embodiments, HyP is designed with at least one FT attached to 3′-end of HyS.

In preferred embodiments, HyP is designed with two FT attached to 5′-end of HyS.

In some embodiments, HyP is designed with two FT attached to 3′-end of HyS.

In some embodiments, HyP is designed with three FT attached to 5′-end of HyS.

In some embodiments, HyP is designed with three FT attached to 3′-end of HyS.

In some embodiments, HyS contains stabilizing moieties such as stabilizing bases, intercalating molecules, MGB, or LNA.

In other aspects, the disclosure describes methods and compositions for hybridization probes with fluorous affinity. Purification of oligonucleotides based on fluorous affinity is known (Fluorous Affinity Purification of Oligonucleotides William H. Pearson, David A. Berry, Patrick Stoy, Kee-Yong Jung, and Anthony D. Sercel The Journal of Organic Chemistry 2005 70 (18), 7114-7122). The cited method is based on introduction of protecting group with fluorous affinity into oligonucleotides during automated synthesis, use the affinity for retention of successfully synthesized chains on the column or a cartridge containing adsorbent with fluorinated carbon surface, elution and subsequent deprotection of the fluorous affinity label. As described herein, it has been found that for the purposes of making hybridization probes, removal of the FT is not needed. As described herein, it has been demonstrated the FT modifications are not interfering with biotin capturing when both FT and biotin tags are present in the probes. It was found that FT are advantageous for the enrichment step in that the FT effectively substitute traditional biotin as an affinity label. Capturing such hybridization probes can be done by a solid support with fluorinated carbon surface or emulsions containing fluorinated carbon chains, as such materials and surfaces have insignificant unspecific interactions with nucleic acids. In some embodiments such FT-containing probes form micelles that effectively hybridize with the targeted nucleic acids and can subsequently be pulled from the mixtures with untargeted nucleic acids. The untargeted nucleic acids can be host DNA or RNA that are usually present in clinical samples before or after preparation of NGS sequencing libraries, before or after PCR amplification. The enrichment success can be measured as a difference in the ratio between targeted and untargeted nucleic acids in the initial mixture and in the mixture after enrichment extraction. Such quantification can be made by comparisons the results using PCR or NGS. Another practical parameter of enrichment is the time needed for the process. The limiting step of most enrichment protocols is time that needed for hybridization of long probes to the targets. Typically, libraries of 80-120-mer DNA or RNA-based probes are used for capturing of potential targets. As described herein, it has been found that shorter probes are hybridizing faster and still tolerant to occasional mutations in the targeted regions. To compensate the loss of binding energy in shorter probes we introduced stabilizing intercalating groups and demonstrated that such probes can function with fluorous tags for capturing the targeted nucleic acids.

As used herein, the term “Hybridizing Sequence” (HyS) refers to an oligo DNA or RNA that includes 20-150 nucleotide sequence that designed to hybridize a region of interest within the target genome. The nucleotides in the sequence are primarily natural but optionally can include unnatural nucleotides with modifications designed to increase binding energy or modulate specificity. Increase in binding energy can be achieved by incorporation of stabilizing bases, intercalating molecules, MGB, or LNA and allows to utilize shorter DNA or RNA to function at temperatures suitable for targeting regions of interest within the target genome.

As used herein, the term “Fluorous” refers to a polyfluorinated carbon chain each comprising 3-30 carbon atoms.

As used herein, the term the term “Intercalator” refers to a small molecule that inserts itself into the structure of DNA. Examples of intercalators include but not limited to acridines and acridiniums, pyrenes, phenazines and phenaziniums, ethidium, psoralens.

As used herein, the term “MGB” or “minor groove binder” refers to a small molecule that insert itself into the minor groove of double stranded structure of DNA. Examples of intercalators include but not limited to Distamycin, Netropsin, Berenil, DAPI, Hoechst, CC-1065, MGB derivative CDPI3 (N-3-carbamoyl-1,2-dihydro-3H-pyrrolo[3,2-e]indole-7-carboxylate tripeptide).

### Hybridization and Hybrid Capture

A variety of methods may be used to enrich for desired sequences from a complex pool of nucleic acids. These methods include the polymerase chain reaction (PCR), molecular inversion probes (MIPs), or sequence capture by hybrid formation (“hybrid capture”). See, for example, Mamanova et al., Nat. Methods 7:111-118 (2010)); U.S. Pat. Publication No. 2014/0031240; and U.S. Pat. Publication No. 2017/0114404.

Next generation sequencing (NGS) applications typically use the hybrid capture method of enrichment. A prepared pool of NGS templates—a library—is heat denatured and mixed with a pool of capture probe oligonucleotides (“hybridization probes”). The probes are designed to hybridize to the regions of interest within the target genome and are usually 60 to 200 bases in length and further are modified to contain a ligand that permits subsequent capture of the bound probes. A common capture method incorporates a biotin group (or groups) on the probes. After hybridization to form the DNA template-probe hybrids is complete, capture is performed with a component having affinity for only the probe. For example, streptavidin-coated magnetic beads can be used to bind the biotin moiety of biotinylated-probes that are hybridized to the desired DNA targets from the library. Washing removes unbound nucleic acids, reducing the complexity of the retained material. The retained material is then eluted from the magnetic beads and introduced into automated sequencing processes.

Though DNA hybridization with the probes can be exquisitely specific, unwanted sequences remain in the enriched pool following completion of the hybrid capture method. The largest fraction of these unwanted sequences is present due to undesired hybridization events between library members having no complementarity to the probes and library members that do (that is, an on-target library member). Two types of sequences lead to undesired hybridizations during hybrid capture methods: (1) highly repetitive DNA elements that are found in endogenous genomic DNA; and (2) the terminal adaptor sequences that are engineered into each of the library members. The repetitive endogenous DNA elements, such as an Alu sequence or Long interspersed nuclear element (LINE) sequence, present in one DNA fragment in the complex pool can hybridize to another similar element present in another unrelated DNA fragment. These fragments, which may originally derive from very different locations within the genome, become linked during the hybridization process of the hybrid capture method. If one of these DNA fragments represents a desired fragment that contains a binding site for a probe, the unwanted fragment will be captured along with the desired fragment. This class of off-target library members can be reduced by adding an excess of the repeat elements to the hybridization buffer of the hybridization reaction. Most commonly, human Cot-I DNA (which binds Alu, LINE, and other repeat sites in the target and blocks the ability of NGS templates to interact with each other on that basis) is added to the hybridization buffer.

Off-target (also referred to as non-target) library members may also be captured due to interactions between terminal adaptor sequences in individual library members.

Typically, library members include a segment of sequence from a gene of interest, for example, a segment for sequencing. If a member is on-target, the sequence from the gene of interest forms a duplex with the capture probe. On-target sequences may include, for example, an exon or an intron (or fragment thereof), a coding region or a non-coding region, an enhancer, an untranslated region, a specific SNP, etc. Typically, library members also include one or more non-target sequences. These non-target sequences typically do not include a target sequence of interest but do include, for example, an adaptor. Because a pool of library members typically will contain at least some identical terminal adaptor sequences, the adaptor sequences are present at a very high effective concentration(s) in the hybridization solution. Consequently, library members containing off-target sequences can anneal to captured target sequences through portions of their appended adaptor sequences, thereby resulting in capture of off-target sequences along with on-target library members. In this way, capture of a single desired fragment can bring along a large number of undesired fragments, reducing the overall efficiency of enrichment for the desired segment. U.S. Pat. Publication No. 2021/0164027 “Compositions and Methods for Improving Library Enrichment” describes methods of mitigation of unspecific binding to the probes including blockers for terminal adaptor sequences. The capturing of hybridized targets is enabled through biotin labeled probes that separated from the mixture with untargeted sequences by streptavidin beads. In some aspects, this disclosure describes methods and compositions for minimizing selection of an off-target nucleic acid by an alternative method of hybrid capture based on fluorous affinity.

In some embodiments, the method includes steps including HyP forming complementary duplexes with targeted nucleic acids. In preferred embodiments, the targeted nucleic acids are libraries of analytes prepared for sequencing. In some embodiments, the method further includes pooling libraries prior to contacting HyP. In some embodiments, the method further includes amplifying the captured sequences after capture. In some embodiments, the method may be used in combination with a blocker oligonucleotide as described in this disclosure. In some embodiments, the method may include the use of a hybridization buffer as described in this disclosure. Hybrid formation performed at temperatures that minimize formation of secondary structures of HyP and targeted nucleic acids. In some embodiments the hybridization temperature is 60°. In preferred embodiments the hybridization temperature is 58°. Hybridization is performed over periods of time ranging from 10 min to 3 hr. In some embodiments, hybridization is performed over 90 min. With shorter HyP comprising 30-mer HyS and stabilizing moieties, hybridization is performed over 10 min. In some embodiments with shorter HyP comprising 40-mer HyS and stabilizing moieties, hybridization is performed over 30 minutes.

Formed hybrids are captured with fluorous surfaces such as PTFE beads, fluorous magnetic beads, fluorous filters or extracted with fluorous liquids. The hybrids adsorbed by fluorous surfaces or extracted by fluorous liquids are washed from any non-hybridized nucleic acids, proteins or PCR inhibitors by the wash buffer. See FIG. 5.

Fluorous- and biotin-based binding affinities are orthogonal, meaning that HyP labeled with both tags can be used simultaneously in the same mixture, hybridizing simultaneously to two different sets of targets over the same hybridization time and in the same reaction volume of the mixture, but then extract targets by using either biotin or fluorous labels therefore enabling separation of corresponding hybrids from the mixture of unhybridized targets and from each other. See FIG. 6.

In some embodiments, biotin-labeled HyP are targeting host nucleic acid targets, such as human genome or mitochondrial nucleic acids, while fluorous-labeled HyP are designed to hybridize with nucleic acids of pathogens therefore providing additional tool for selective enrichment of the targeted nucleic acid by selective removal of host nucleic acids.

In some embodiments, fluorous-labeled HyP are targeting host nucleic acid targets, while biotin-labeled HyP are designed to hybridize with nucleic acids of pathogens for selective removal of host nucleic acids.

In some embodiments, biotin-labeled HyP are targeting the first set of nucleic acid of pathogen targets, while fluorous-labeled HyP are designed to target the second set of nucleic acids of pathogens. In such embodiment, the host nucleic acid is not designed to hybridize with either set of probes and can be largely removed by wash from streptavidin bound and fluorous bound HyP that bind and hold nucleic acid targets of interest as double stranded hybrid. The affinity supports for the two different tags can be made separable: for example, Streptavidin magnetic beads and PTFE beads, where the first type pulled with a magnet, and the second precipitated by centrifuge or filtered off.

Such orthogonal labeling of probes and subsequent separation of streptavidin-bound and fluorous-bound targeted nucleic acids enables selective sequencing of targets of interests as two different sets expanding useful research and clinical applications of metagenomics.

In some embodiments, the HyP may contain simultaneously FT and biotin label. Such HyP after hybridization with the targeted sequence can be captured by either fluorous surface or extracted by fluorous liquid, or captured by streptavidin surface (e.g., SA magnetic bead), or captured by a combined streptavidin fluorous surface.

### Fluorous Hybridization Probes

Fluorous Hybridization Probes comprising oligonucleotides having HyS and FT can be synthesized using automated oligo synthesizer and reagents further described in examples. FT on 5′-end is preferred, as it can be used for affinity purification of successful oligos containing full HyS. Methods for affinity purification of oligonucleotides using protecting groups with fluorous affinity are described in U.S. Pat. Publication No. 2006/0178507. The inventors have found that fluorous affinity groups do not interfere with hybridization under enrichment hybridization conditions, and therefore these affinity group do not have to be removed. Further, the same affinity phenomenon was used to capture hybridized probes to fluorous solid supports or extract with fluorous liquids. Fluorous surfaces have minimal non-specific affinity to both hydrophobic and hydrophilic molecules, and presumably bind only to fluorinated molecules. This aspect allowed us to maximize capturing of FT-containing probes and their hybrids with the targeted sequences and at the same time to minimize unspecific binding of all other biomolecules during capturing process. Once hybridization is complete and a DNA template-probe hybrid is formed, a capture means (that is, a component having affinity for the probe including, for example, a fluorous-coated magnetic bead) is used to bind the probe that is hybridized to the DNA target, removing the target from a pool of oligonucleotides. The capture probe consists of 20-200 nucleotides. In preferred embodiments the capture probe consists of 40-80 nucleotides. All capture probes contain at least one FT. Capture probe may contain several FT, typically but not limited to two or three on the 5′- or on the 3′-end. Some designs may have up to three FT on each 3′- and 5′-end of the probe making up to six FT per the probe molecule.

HyP with single FT on the 5′-end can be synthesized using oligo synthesizer and standard protocol terminating the synthesis with reagents FT1-PA or FT2-PA providing FT1 or FT2 tags at the 5′-end of HyP. 3′-end can be free by using universal CPG or blocked typically with C3 propanol group by using 3′-Spacer C3 CPG from AM Chemicals LLC, 4065 Oceanside Blvd., Suite M Oceanside, CA 92056-5824.

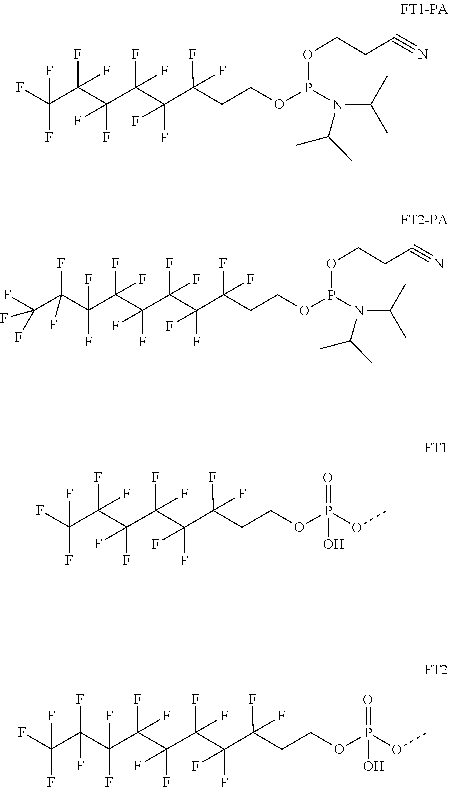

The probes with FT1 or FT2 and an optional 3′-C3 spacer are having the following structure:

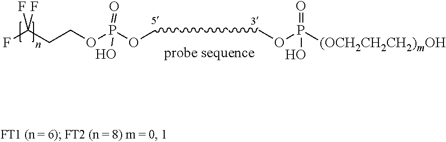

HyP with two FT on the 5′-end can be synthesized using oligo synthesizer and protocol recommended for Symmetric Doubler Phosphoramidite and terminating the synthesis with reagents FT1 or FT2. The Doubler is commercially available from Glen Research, cat. no. 10-1920-02.

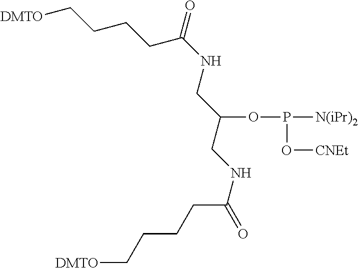

### Doubler Phosphoramidite

The probes with the Doubler and an optional 3′-C3 spacer are having the following structure:

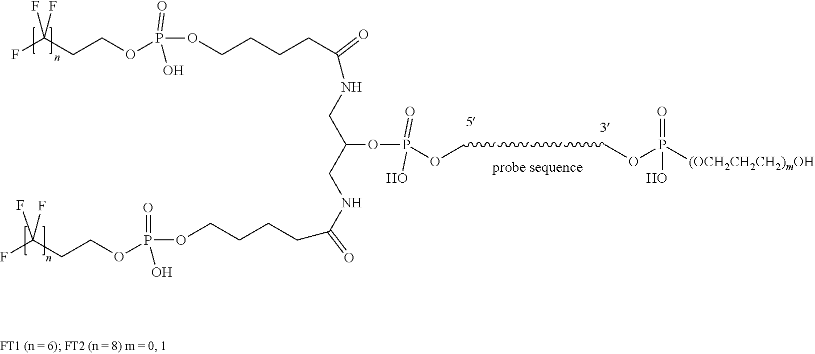

Similarly, HyP with tree FT on the 5′-end can be synthesized using oligo synthesizer and protocol recommended for Trebler Phosphoramidite and terminating the synthesis with reagents FT1 or FT2. Trebler phosphoramidite (cat. no. 10-1922-02) and Long Trebler phosphoramidite (cat. no. 10-1925-90) are commercially available from Glen Research.

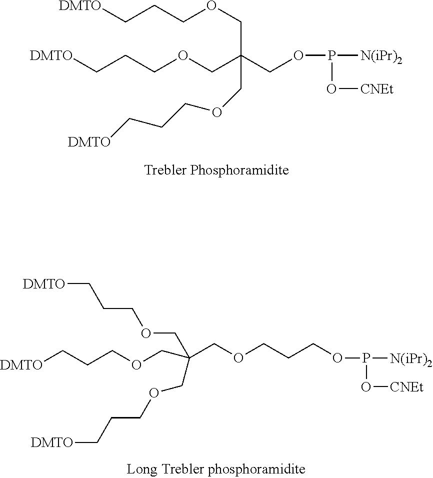

The probes with the Trebler or Long Trebler and an optional 3′-C3 spacer are having the following structure:

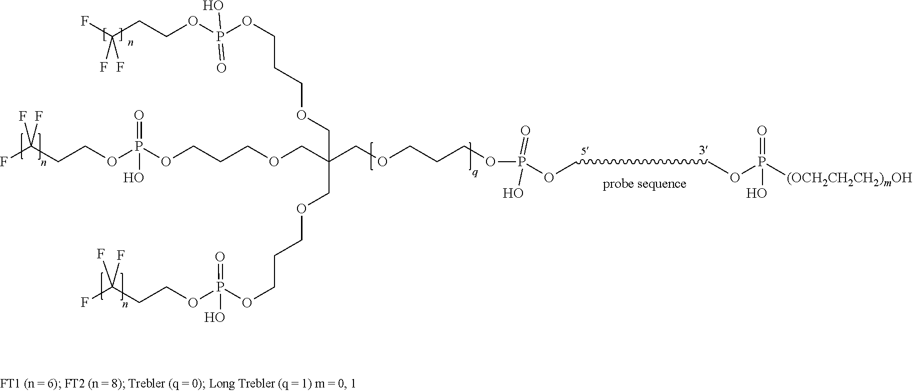

HyP with single FT on the 3′-end can be synthesized using oligo synthesizer and standard protocol starting with the Asymmetric Doubler (Lev) Phosphoramidite. The reagent is commercially available from Glen Research, cat. no. 10-1981-02. The levulinyl protecting group can be selectively removed without cleavage of the oligonucleotide from the CPG by treatment with 0.5M Hydrazine hydrate in 1:1 pyridine/acetic acid. Terminating the synthesis with reagents FT1-PA or FT2-PA after selective removal of levulinyl protecting group provides FT1 or FT2 at the 3′-end of the probe sequence. The very terminal 3′-end of HyP can be free by using universal CPG or blocked typically with C3 propanol group by using 3′-Spacer C3 CPG. 5′-end can be free (OH group) or terminated with a phosphoramidite of any desired group such another FT, or set of FT, or labels such as biotin or fluorescent dye by using appropriate phosphoramidite, for example 5′-Biotin Phosphoramidite (Glen Research cat. no. 10-5950-02), or 5′-Fluorescein Phosphoramidite (Glen Research cat. no. 10-5901-02).

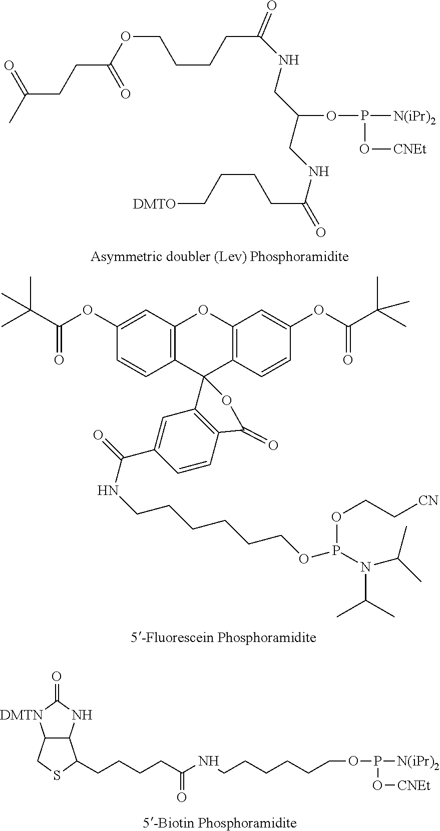

The synthesis is performed according to the following protocol:
1. Couple Asymmetric Doubler to CPG (Universal or 3′-Spacer C3)
2. Build the probe sequence
3. Terminate 5′-end with a phosphoramidite of a selected ligand L (e.g., FT, Biotin or FAM)
4. Selectively remove the levulinyl protecting group without cleavage of the oligonucleotide from the CPG
5. Wash with acetonitrile
6. Couple with CF1-PA or CF2-PA

Alternatively, FT-modified CPG is made first by introduction of Asymmetric Doubler, selective deprotection of levulinyl group, coupling with FT and then using that FT-containing CPG for building libraries of HyP. The synthesis is performed according to the following protocol:
1. Couple Asymmetric Doubler to CPG (Universal or 3′-Spacer C3)
2. Selectively remove the levulinyl protecting group without cleavage of the oligonucleotide from the CPG
3. Couple with CF1-PA or CF2-PA
4. Wash with acetonitrile
5. Split CPG to different columns or plate
6. Build the library of probe sequences in different columns or wells
7. Terminate 5′-end with a phosphoramidite of a selected ligand L (e.g., FT, Biotin or FAM)

The desired HyP that are made by both protocols have the following structure:

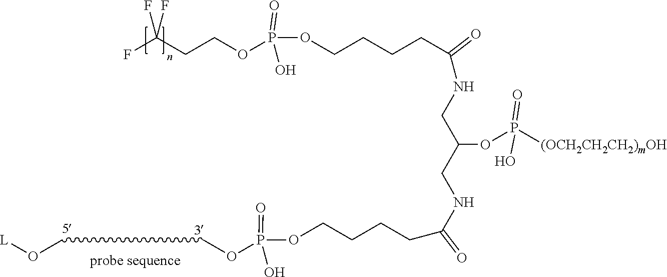

Where FT1 (n=6) and FT2 (n=8); m=0, 1 and L=FT1, FT2, Biotin label or fluorescent dye label. In some embodiments, fluorescent dye may comprise FT, such as coumarin dye that can be introduced into HyP using phosphoramidite Coumarin-FT-PA. The reagent allows simultaneous introduction of fluorescent label and FT into HyP.

In some embodiments, Asymmetric Doubler is used on the 5′-end or simultaneously on the 3′- and the 5′-ends providing flexibility in the design of HyP with any number of FT at any terminal position and in adding desired ligands L to either end.

Positioning of FT is not limited to terminal ends of HyP. Phosphoramidite reagents are known for introduction of levulinyl moieties to internal positions of oligos during automated synthesis. For example, Glen Research's 5-Me-dC Brancher Phosphoramidite (cat. no. 10-1018-02) or phosphoramidite reagent based on 2′-O-(2-Levulinyl-hydroxyethyl)-uridine (2′-OLev-U) (Madhavaiah Chandra, et el., A modified uridine for the synthesis of branched DNA. Tetrahedron; 63 (2007), 35.-S. 8576-8580).

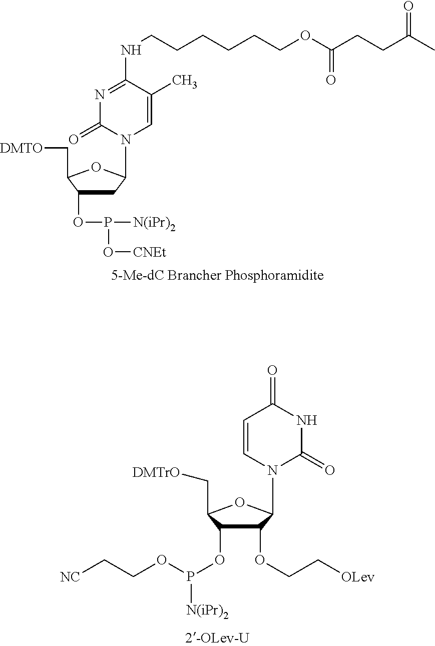

Certain applications may benefit from FT-containing HyP that are comprising a fluorescent dye. Fluorescence of such probes can be modulated by change in hydrophobicity of the environment. The brightness of the dye is modulated by polarity of the environment providing a tool for detection of a physical state of the HyP. Therefore, such probes can be useful both as targeted nucleic acid enrichment tools and as an indicator of micelle formation or interaction with hydrophobic membrane.

This disclosure provides a method of synthesis and application of a fluorescent coumarin dye Coumarin-FT-PA, that contains two FT. The reagent can be used for simultaneous introduction of fluorescent label and FT into HyP by a standard protocol using automated synthesizer.

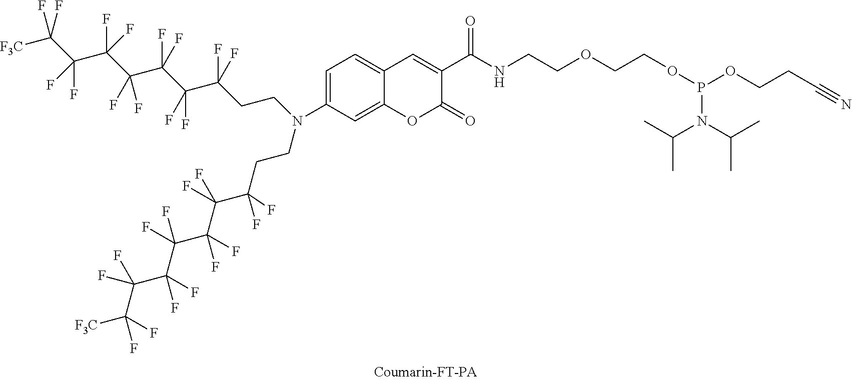 [SMILES: CC(C)N(C(C)C)P(OCCC#N)OCCOCCNC(=O)c1cc2ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc2oc1=O]

HyP containing more than two FT can be made by using phosphoramidites containing two FT in the molecule. Such reagents as protective groups have been demonstrated for affinity purifications of oligonucleotides with subsequent removal by deprotection (e.g. Christian Beller, Willi Bannwarth Helvetica Chimica Acta 2005 Vol. 88; Iss. 1, p. 171-179). Here are disclosed several reagents that can introduce two symmetrical FT into HyP during automated oligo synthesis and retain the FT on HyP in a chemically stable form without removal. pIA-2FT is synthesized from p-Iodoaniline, pAPA6-2FT, pAPA8-2FT, from p-Aminophenethyl alcohol, m-AP-2FT from m-Aminophenol and pAB-2FT is based on p-Aminobenzaldehyde intermediate. The reagents contain two 1H,1H,2H,2H-Perfluorooctyl tags. In preferred embodiments, these reagents will terminate 5′-end of HyP during automated synthesis, and fluorous affinity of FT is used for purification of fluorous cartridge such as Fluoro-Pak™ (#FP-7210) and Fluoro-Pak™ H Columns (#FP-7220) from Berry&Associates. The same affinity tags are used for retention of hybridized targeted nucleic acid in the enrichment process.

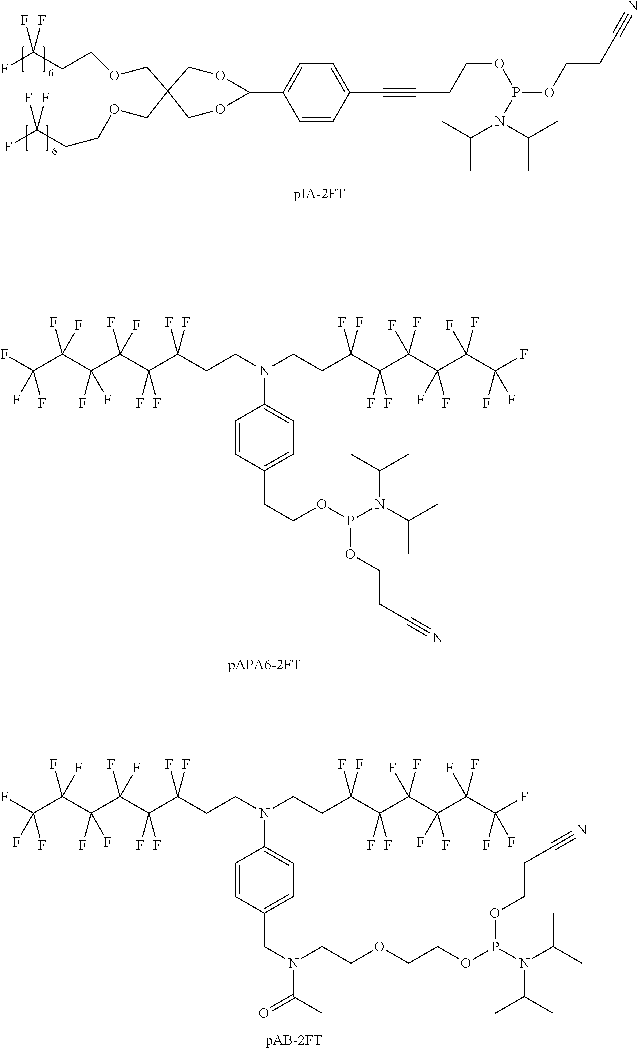

### HyP Containing Stabilizers

A hybridization process using a 80-200 nucleotides long HyP requires at least 90 min incubation with samples containing targeted nucleic acids. Typical hybridization temperature is 580. The temperature and hybridization buffers are optimized for target availability and binding with multiple probes in the library. Shortening the probes would accelerate binding, but hybridization will require lower temperature at which targeted nucleic acids may fold into secondary structures and become unavailable for hybridization. Many methods of increasing binding temperature during hybridization are known. To compensate for a loss of binding energy, the shorter probes are designed with stabilizing moieties such as intercalators or minor groove binders (MGB). Typical probe is designed with one or two intercalating units or MGB at the terminal 3′- and 5′-ends of the probe. Further increase of binding can be achieved by introduction of additional intercalating moieties to internal positions of the probes. MGB and intercalating units can be introduced to the probes during automated oligo synthesis or post synthetically using conjugation chemistries or by combination of both approaches. Stabilizing bases such as modified thymine (U.S. Pat. No. 9,598,455), modified cytosine (U.S. Pat. No. 9,598,456) can be introduced to internal positions of oligos using corresponding phosphoramidites. Minor groove binding stabilizers can be introduced to terminal positions of oligos by using reagents from Glen Research MGB-CPG (CDPI3 MGB™ CPG, cat. no. 20-5924-13) or 5′-CDPI3 MGB™ Phosphoramidite (cat. no. 10-5924-95).

The current disclosure demonstrates applications of shorter HyP with stabilizers that compensate the loss of binding capacity of the shorter duplex hybrids at a standard 58° temperature. Shorter probes hybridize faster and allow to reduce overall time-to-result. Stabilizers have low sequence specificity that helps shorter probes to tolerate some level of mismatches in the targeted nucleic acids.

Short hybridization probes are designed with affinity groups biotin or FT or combination of thereof. For example, hybridization duplexes can be stabilized by pyrene moieties (2′-Pyrene modified oligonucleotide provides a highly sensitive fluorescent probe of RNA. Yamana K, Twase R, Furutani S, Tsuchida H, Zako H, Yamaoka T, Murakami A. Nucleic Acids Res. 1999 Jun. 1; 27(11):2387-92) or MGB (Kutyavin T, Lokhov S, Lukhtanov E, Reed M W. Chemistry of minor groove binder-oligonucleotide conjugates. Curr Protoc Nucleic Acid Chem. 2003 August; Chapter 8:Unit 8.4). We demonstrated utility of short hybridization probes using known N-(2-hydroxyethyl)phenazinium intercalating stabilizer (Synthesis and high stability of complementary complexes of N-(2-hydroxyethyl)phenazinium derivatives of oligonucleotides. S. G. Lokhov, M. A. Podyminogin, D. S. Sergeev, V. N. Sil'nikov, I. V. Kutyavin, G. V. Shishkin, V. P. Zarytova. S. G. Lokhov, M. A. Podyminogin, D. S. Sergeev, V. N. Sil'nikov, I. V. Kutyavin, G. V. Shishkin, V. P. Zarytova. Bioconjugate Chem. 1992, 3, 5, 414-419). The N-(2-hydroxyethyl)phenazinium moiety is introduced into oligonucleotides through linkers containing primary amino group by conjugation with N-(2-hydroxyethyl)phenazinium chloride (Phe). In a preferred embodiment, the HyP contains two 3′-, and 5′-terminal Phe intercalating groups and two 5′-FT. Amino linker can be introduced to the 3′-end using Glen Research reagents 3′-Amino-Modifier C7 CPG 1000 (cat. no. 20-2958-13), and to the 5′-end with Amino-Modifier Serinol Phosphoramidite (cat. no. 10-1997-02).

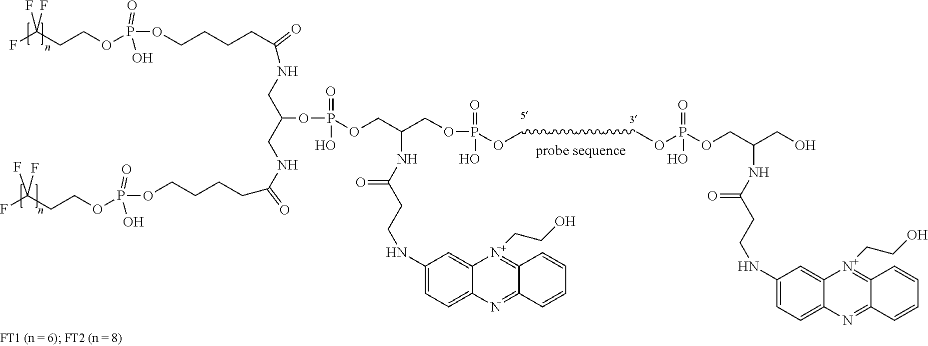

While direct method of introduction of FT into HyP using corresponding phosphoramidites and automated synthesis is preferred, alternative conjugation methods offer flexibility by enabling preparation of libraries by automated oligo synthesis and then transforming them into FT-containing HyP libraries using post-synthetic conjugation with FT-containing reagents for fluorous affinity enrichment. Such conjugation can be performed with the entire library at once, and the library can be purified using fluorous affinity columns. Many oligo-compatible conjugation chemistries can be used. The methods include but not limited to azido-terminal alkyne coupling (Click chemistry), hydrazides and hydroxylamines coupling to oligos containing aldehyde and ketone reactive groups. Alkyne modifiers are used to react with azide-labeled functional groups to form stable bonds through the Click reaction. 5′ Hexynyl is one way to introduce a 5′ terminal alkyne group. 5-Octadinynyl dU is a modified base with an 8-carbon linker terminating in an alkyne group and is the preferred way to insert alkynes at internal positions within a sequence. This modification is also available for 3′ or 5′ attachment. Oligos with such modifications are commercially available from integrated DNA Technologies, Inc

### Azide-Modified CPG is Available at Primetech (Cat. No. 0058-500/0058-1000)

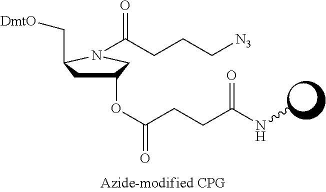

Azido-containing FT can be prepared from FT1-PA or FT2-PA and Azide-modified CPG in one step using oligo synthesizer. The reagents can be used for subsequent post-synthetic conjugation with alkynyl oligos by Click chemistry with a copper catalyst.

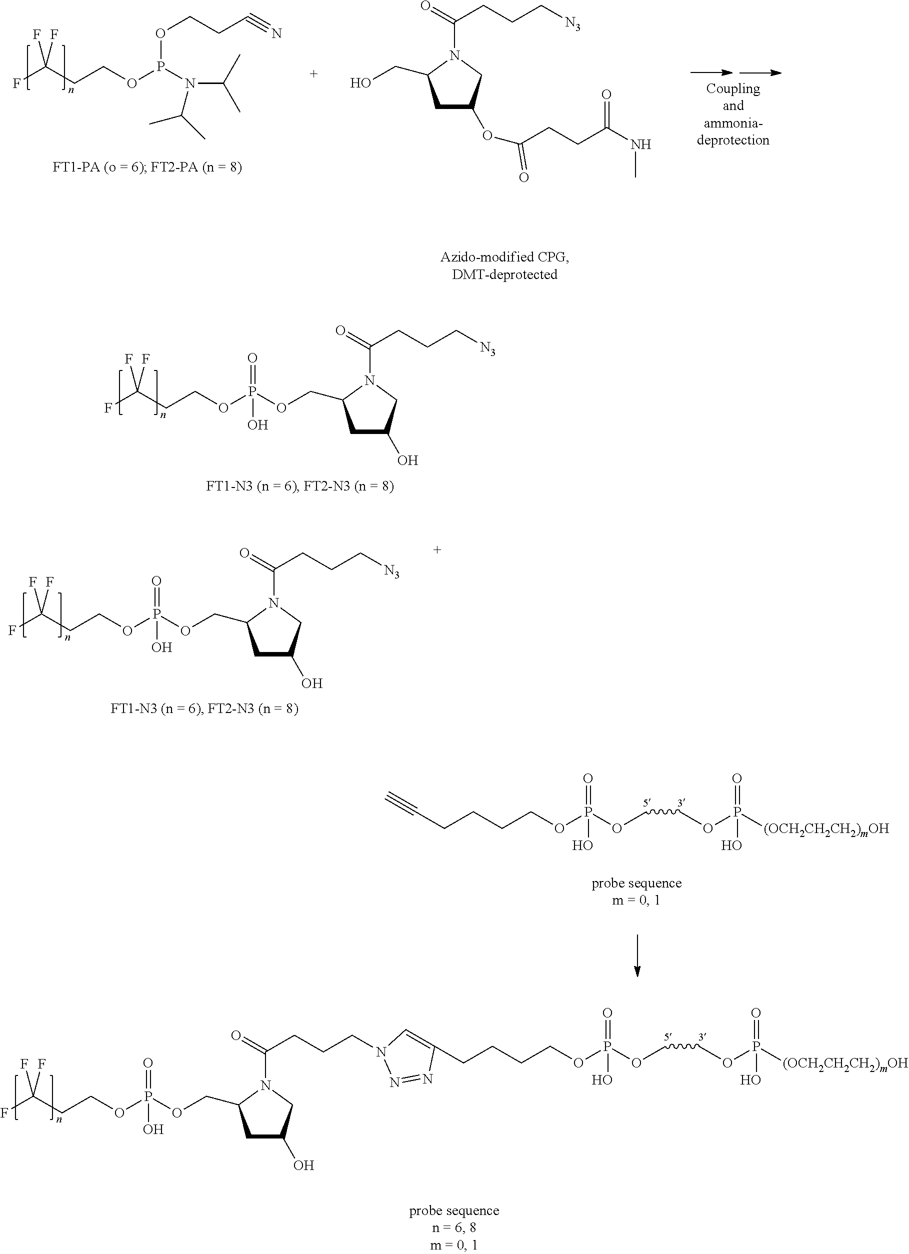

A similar approach based on subsequent introduction of the Symmetric Doubler and then FT1-PA or FT2-PA provides azido reagent for simultaneous introduction of two FT into the HyP. Such an azide reagent has multiple phosphate groups that provide water solubility of the reagent for Click chemistry coupling with alkyne-oligo. The following structure is an illustration of such design.

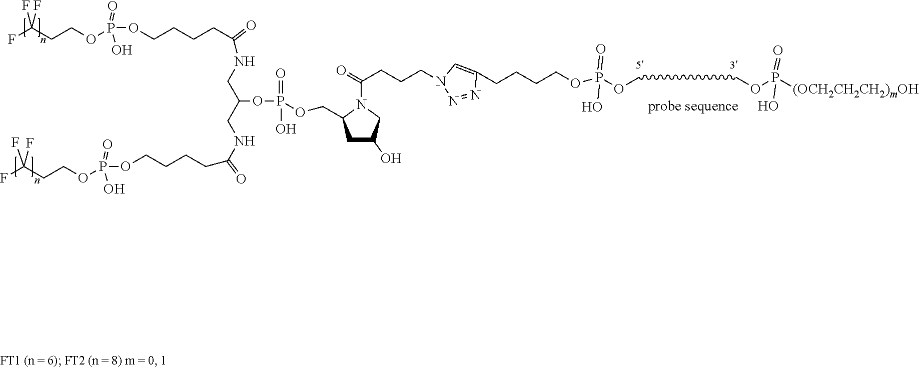

One similar method is applicable to conjugation of multiple alkyne-containing oligo libraries providing FT-containing HyP libraries.

Other conjugation methods disclosed include reactions between aldehyde- and ketone-containing oligonucleotides and water-soluble hydrazide or hydroxylamine derivatives of FT. Synthesis methods of such reagents are provided in the Examples section.

Methods of synthesis of ketone Ket1-PA and Ket2-PA and aldehyde Ald-PA phosphoramidite reagents are presented in examples. These reagents allow introduction of aldehyde and ketone groups into oligonucleotides during oligo synthesis. The aldehyde reagent Ald-PA is protected in acetal form. The acetal can be used as a hydrophobic moiety for Glen-Pak cartridge purification using Glen-Pak DNA Purification Cartridge (60-5100-XX, 60-5200-XX) after automated synthesis. It requires acid deprotection under standard detritylation conditions.

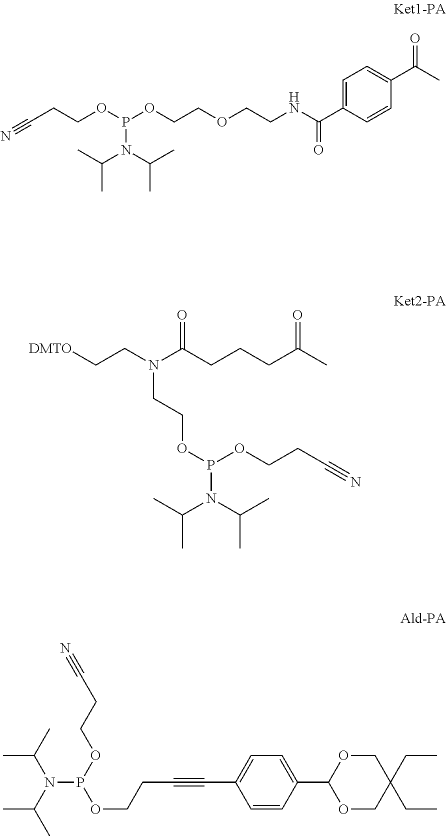

The following examples are provided for the purpose of illustrating, not limiting, embodiments of the invention.

### EXAMPLES

Reagents FT1-PA (n=6) and FT2-PA (n=8) are synthesized according to the general procedure for compound FT2-PA (Flynn, G. E., et al., Reversible DNA micro-patterning using the fluorous effect. Chemical Communications, 2017, vol. 53, #21, p. 3094-3097).

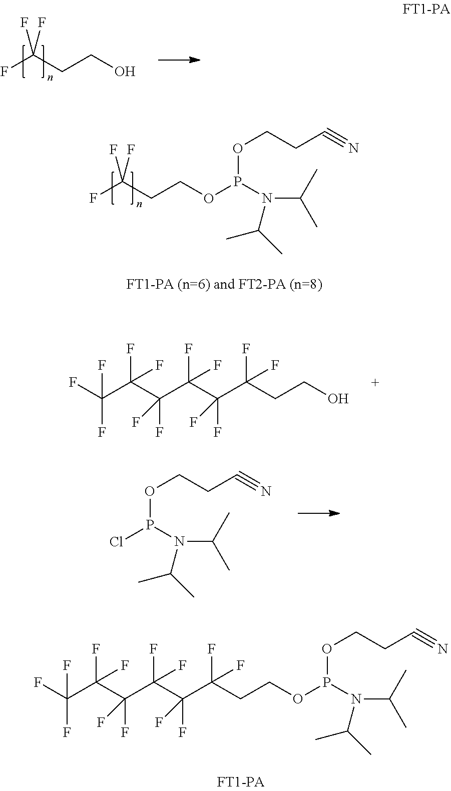

To a solution of perfluorooctanol (5.0 g, 13.7 mmol) in anhydrous acetonitrile (106 mL) is added diisopropylethylamine (4.3 mL, 25 mmol), followed by dropwise addition of 2-Cyanoethyl N,N-diisopropylchlorophosphoramidite (5.0 g, 21 mmol). The RM is allowed to sit at ambient temp for 24 hr. RM is then concentrated in vacuo, diluted with EtOAc, washed with sat. NaHCO₃, brine, dried over Na₂SO₄ and concentrated in vacuo giving a clear and colorless oil. Crude residue is azeotroped with toluene (2×10 mL), before concentrating to a constant weight. Purification via flash chromatography (0-10% EtOAc/heptane on deactivated silica (5% Et₃N)) provides the product (5.73 g, 74%) as a clear and colorless oil. TLC 75% EtAOc/Heptane Rf 0.73.

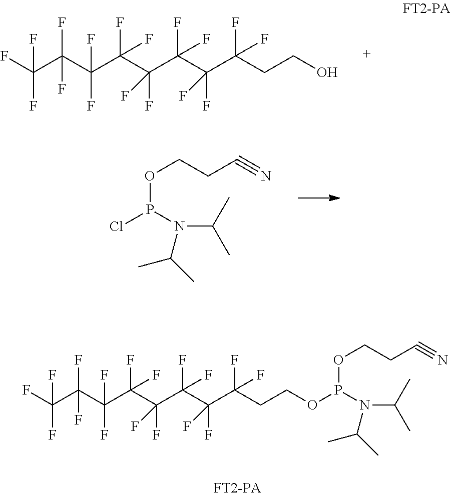 [SMILES: OCCC(F)(F)C(F)(F)C(F)(F)C(F)(F)C(F)(F)C(F)(F)C(F)(F)C(F)(F)F ; CC(C)N(C(C)C)P(Cl)OCCC#N ; CC(C)N(C(C)C)P(OCCC#N)OCCC(F)(F)C(F)(F)C(F)(F)C(F)(F)C(F)(F)C(F)(F)C(F)(F)C(F)(F)F]

To a solution of perfluorodecanol (10.01 g, 21.5 mmol) in anhydrous acetonitrile (24 mL) was added diisopropylethylamine (5.4 mL, 30.8 mmol, 1.4 eq), followed by dropwise addition of 2-Cyanoethyl N,N-diisopropylchlorophosphoramidite (5.1 mL, 23 mmol, 1.1 eq). The RM is allowed to sit at ambient temp for 48 hr. RM was concentrated in vacuo and purified via flash chromatography (100% Toluene on deactivated silica (5% Et₃N)) giving the product (11.42 g, 82%) as a clear and colorless oil. TLC 20% EtOAc/heptane Rf 0.4.

Examples of compounds with two FT with the following general structure:

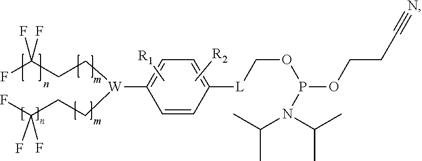

Where n=5-18, m=1, 2, W is a linking group 1-10 atoms C3-C20 heteroalkylene comprising 1-6 heteroatoms selected from P, O, N, S, and combinations thereof.

Phosphoramidites pIA-2FTa and pIA-2FTb are synthesized by the following procedures:

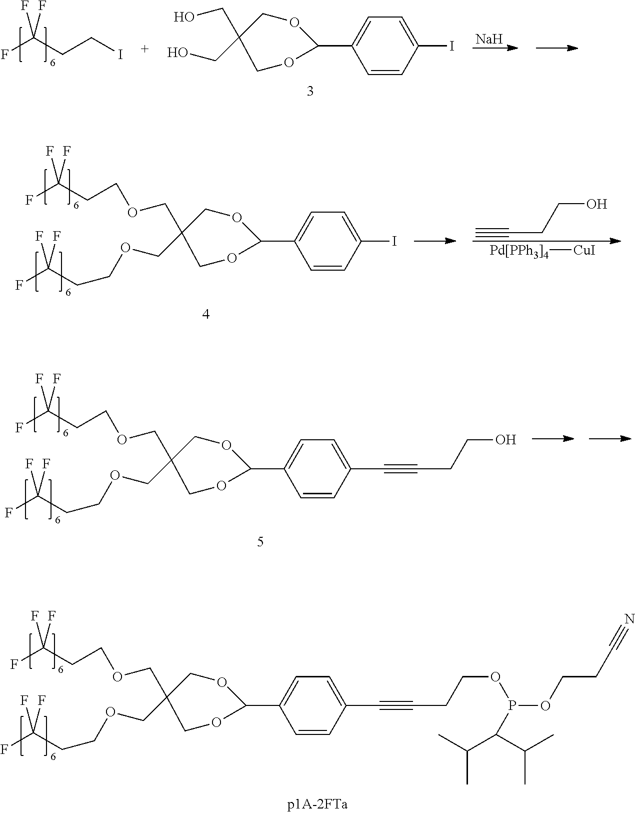

### Compound 3

Pentaerythritol (3.27 g, 24 mmol, 1.2 eq) and 4-iodobenzaldehyde (4.91 g, 21.2 mmol, 1 eq) are heated in xylenes in the presence of camphorsulfonic acid (0.49 g, 2 mmol, 0.1 eq) on a rotovap water bath at 90° and slowly evaporated. Fresh portion of xylenes added and evaporated again. The reaction mixture is then concentrated in vacuo and redissolved into EtOAc. The organic layer is washed with sat. NaHCO₃, brine, dried over Na₂SO₄ and concentrated in vacuo giving a white wax.

### Compound 4

Compound 3 (30 g, 0.09 mmol) in dry DMF (600 mL) is treated with 80% NaH in oil (8.6 g, 0.22 mmol) and stirred with ice cooling for 1 hr. 1H,1H,2H,2H-Perfluorooctyl iodide (104 g, 0.22 mmol) is introduced slowly via syringe, and the reaction mixture continued stirring on ice for another hour. If TLC showed starting diol or mono-alkylated intermediate still present, another portion of 80% NaH (4.3 g, 0.11 mmol) is added, and after 30 min, another portion of 1H,1H,2H,2H-Perfluorooctyl iodide (52 g, 0.11 mmol) is added while continue stirring on ice.

The product is isolated by flash chromatography using DCM-Heptane mixture. Compound 4 is obtained.

Compound 5 is made by following general procedure for Sonogashira coupling.

Copper iodide (0.327 g, 1.72 mmol, 0.2 eq), Pd(PPh₃)₄ (0.993 g, 0.86 mmol, 0.1 eq) and the aryl iodide (9.0 g, 8.6 mmol, 1 eq) are combined in flask with stir bar. The flask is placed on high vacuum overnight and the round bottom is refilled with argon. The solids are dissolved with DMF (17 mL) and Et₃N (2.39 mL, 17.2 mmol, 2 eq) is added. 3-Butyne-1-ol (0.779 mL, 10.3 mmol, 1.2 eq) is then added dropwise and the RM stirred at ambient temp for 3 hr. RM is then diluted with 60 mL of 0.1M Na₂EDTA and 150 mL of EtOAc, stirred for 30 min and transferred to a separatory funnel with 300 mL of EtOAc. Organic layer washed with 0.1M Na₂EDTA, sat. NaHCO₃, brine, dried over Na₂SO₄ and concentrated in vacuo giving a dark brown/reddish oil. Purification via flash chromatograph gives the product as a light-yellow solid. TLC is performed in 60% EtOAc/heptane.

Phosphoramidite pIA-2FTa is made by following general procedure for compound FT1-PA.

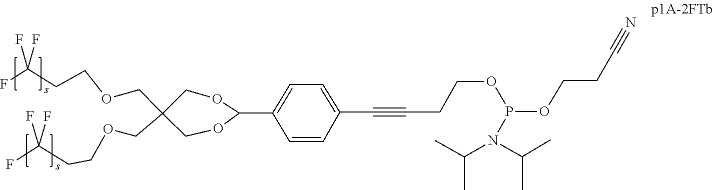

Phosphoramidite pIA-2FTb is made by following general procedure for compound pIA-2FTa starting from compound 3 and 1H,1H,2H,2H-Perfluorodecyl iodide.

Reagent based on p-aminobenzaldehyde:

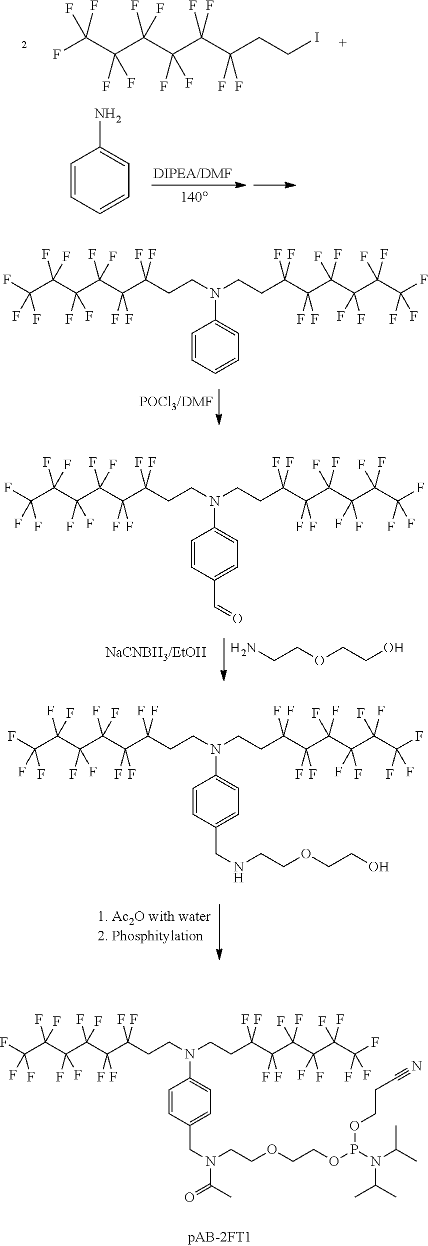

Reagent based on p-aminophenethyl alcohol:

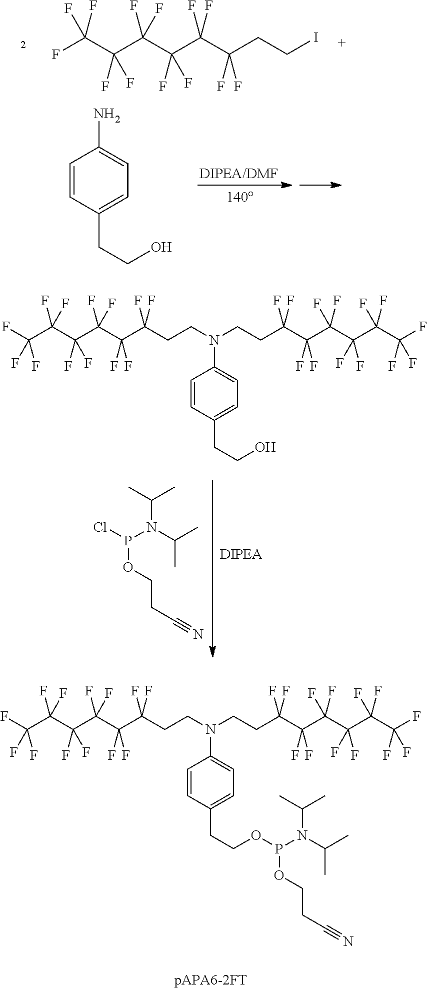

Analogously, starting with 4,4,5,5,6,6,7,7,8,8,9,9,10,10,11,11,11-Heptadecafluoroundecyl iodide, obtained pAPA8-2FT, a tag reagent with a longer FT chain:

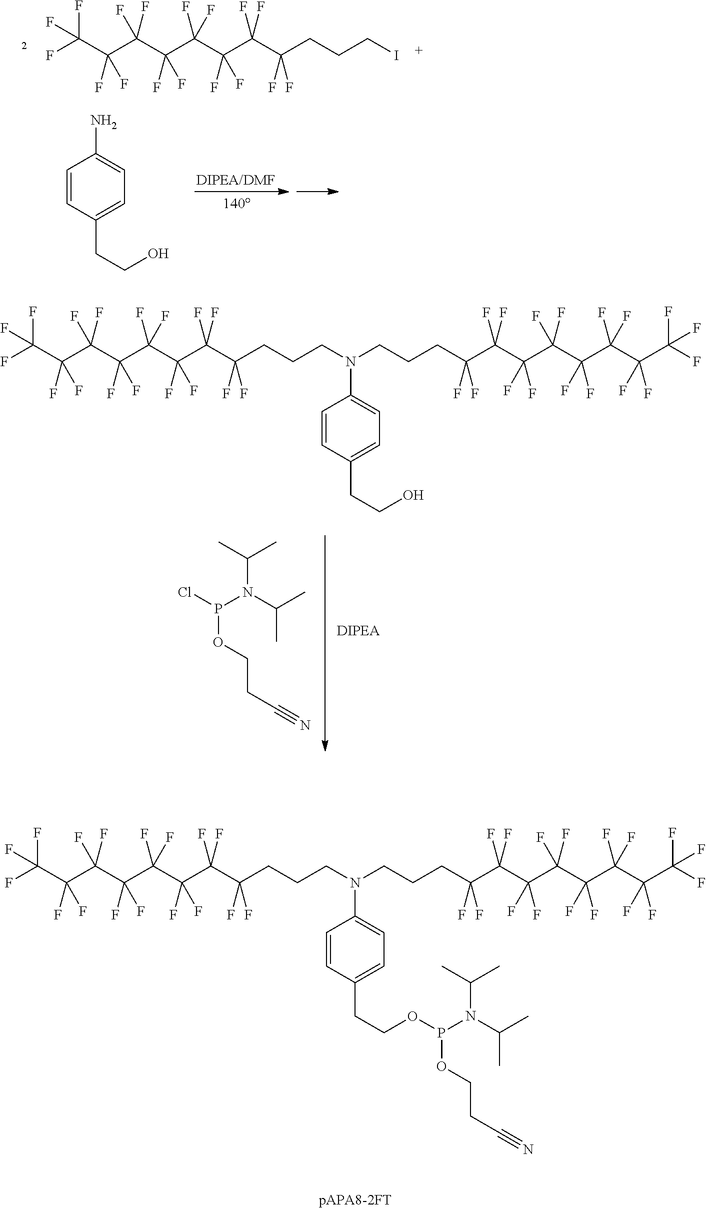

Reagent based on p-iodoaniline:

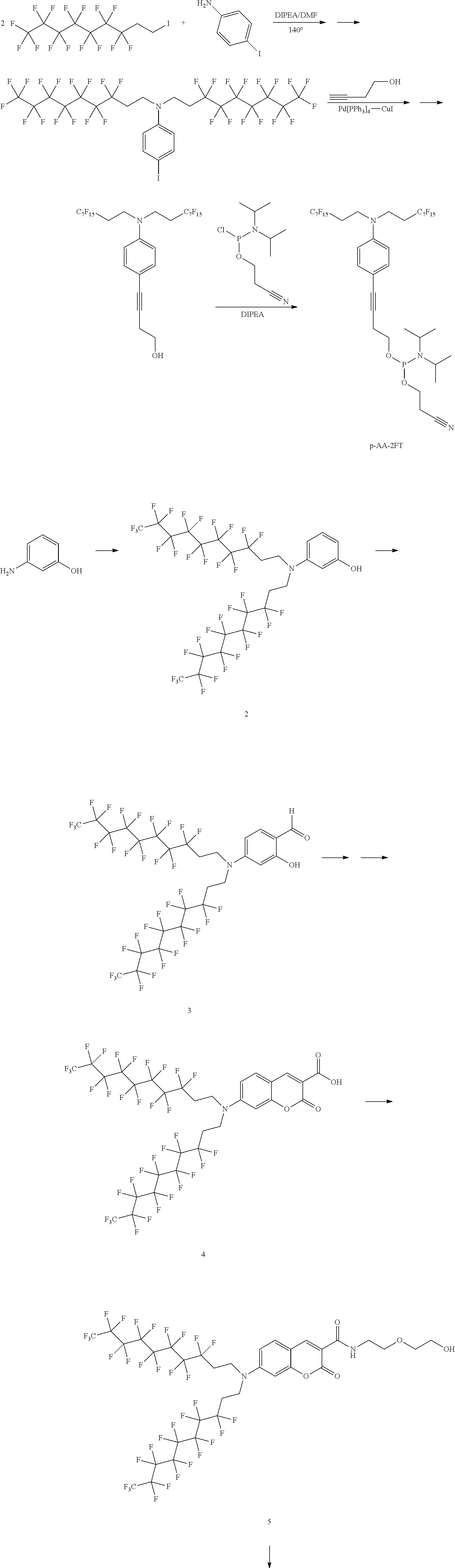

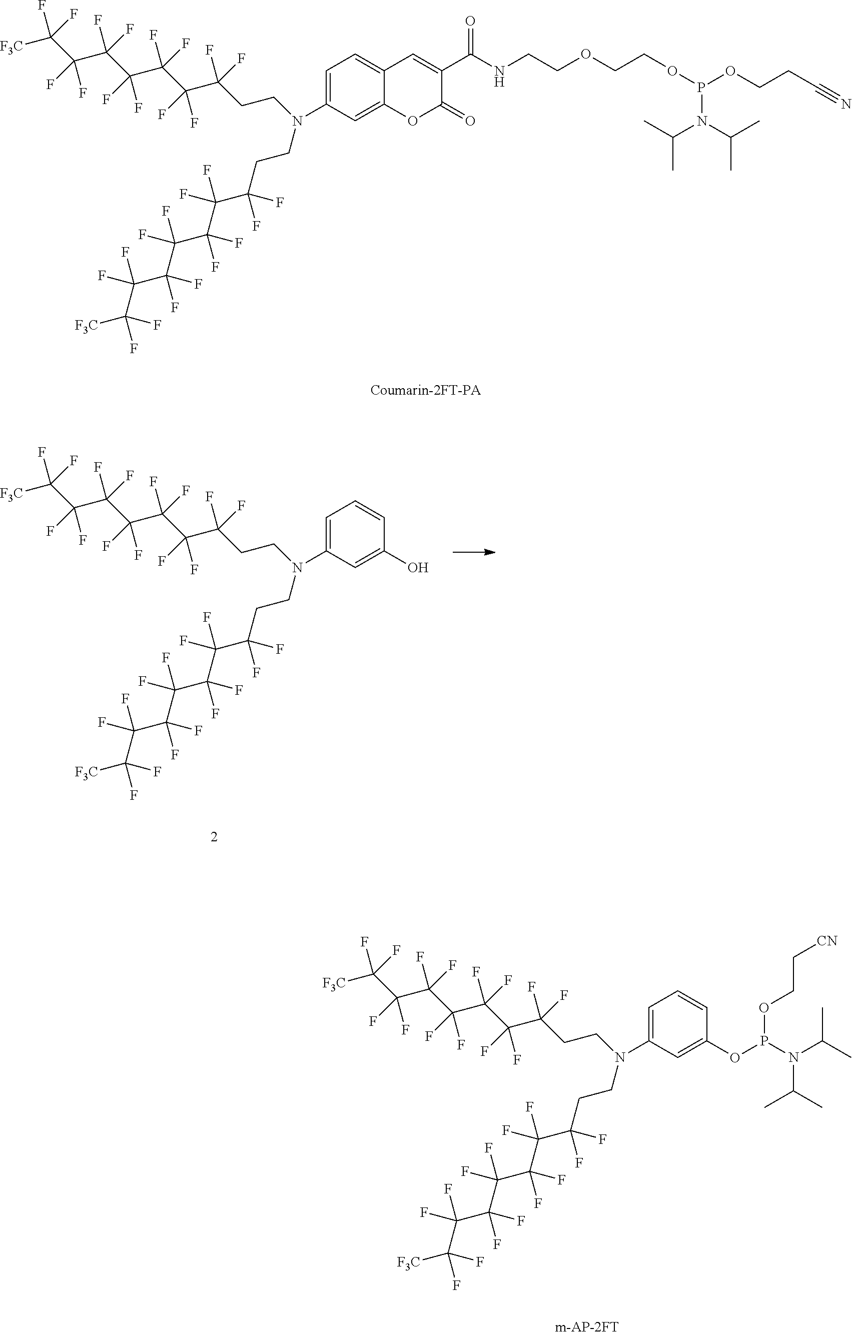 [SMILES: CC(C)N(C(C)C)P(OCCC#N)OCCOCCNC(=O)c1cc2ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc2oc1=O ; Oc1cccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)c1 ; CC(C)N(C(C)C)P(OCCC#N)Oc1cccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)c1]

### Representative Procedures

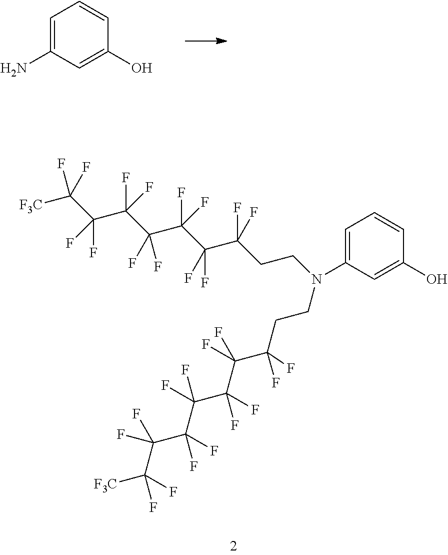 [SMILES: Nc1cccc(O)c1 ; Oc1cccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)c1]

(2). 1H,1H,2H,2H-Perfluorodecyl iodide (9.7 g Iodide, 16.8 mmol, 4 eq), 3-aminophenol (0.502 g, 4.6 mmol, 1 eq) and DIPEA (3.8 mL, 21 mmol, 5 eq) are mixed at room temp with 5.0 mL of DMP in a sealed tube under Ar. The reaction mixture is heated at 180° for 3 days and monitored by TLC until completion. The reaction is cooled to room temperature, diluted with water (100 mL) and EtOAc (100 mL), and the organic layer is washed with sat. NaHCO₃ (100 mL), brine (50 mL), dried over Na₂SO₄ and concentrated in vacuo. The crude mixture is then purified via flash chromatography giving amino phenol (2) as a pure compound.

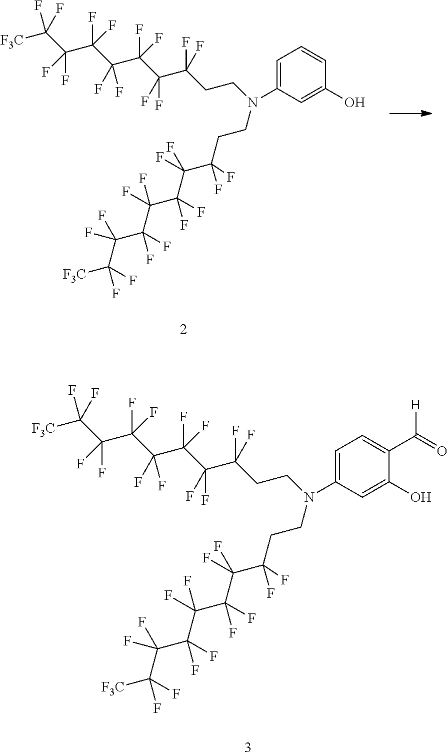 [SMILES: Oc1cccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)c1 ; O=Cc1ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc1O]

(3) POCl₃ (1.3 mL, 13.8 mmol, 3 eq) and DMF (1.8 mL, 23 mmol, 5 eq) are cooled on solid dry ice in separate round bottoms under Ar. The DMF is then poured onto the solid POCl₃ and swirled while warming to room temp. The Vilsmeier regent is then poured onto amino phenol (2) neat and heated to 75° for 2 hours. The reaction is quenched with 60 mL H₂O, extracted with EtOAc (2×50 mL), washed with brine/bicarb (1:1, 50 mL), dried over Na₂SO₄, and filtered over a pad of silica with 1:1 EtOAc/heptane, giving pure (3)

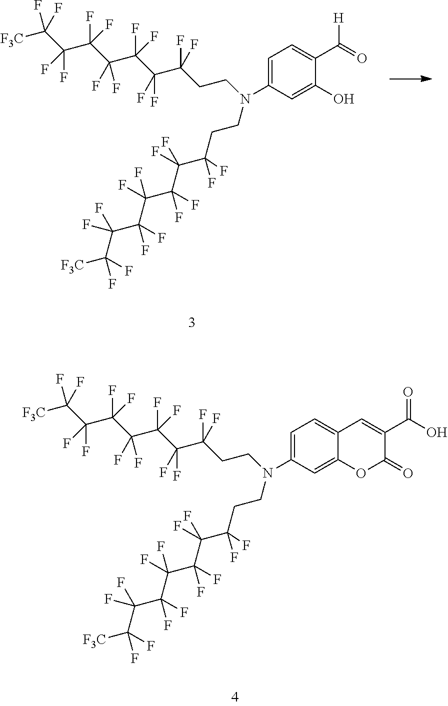 [SMILES: O=Cc1ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc1O ; O=C(O)c1cc2ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc2oc1=O]

(4) Diethyl malonate (1.0 mL, 6.5 mmol, 1.3 eq) and morpholine (4.3 mL, 5 mmol, 1 eq) is added to a solution of aldehyde (3) (5.14 g, 5 mmol, 1 eq) in 3.3 mL of ethanol. The reaction mixture is stirred under reflux for 2 h, diluted with water (50 mL), extracted with ethyl acetate (3×50 mL), dried over Na₂SO₄ and concentrated in vacuo. The crude mixture is then dissolved into 10 mL of absolute ethanol, and 10 mL of a 10% NaOH solution is added. The reaction is refluxed at 95° for 2 hours and monitored by TLC for completion. After the reaction is over, the solution is cooled to room temperature, and 1.0 M HCl is added dropwise until a pH of 2 is achieved at which point the product will precipitate out of solution giving carboxylic acid 4.

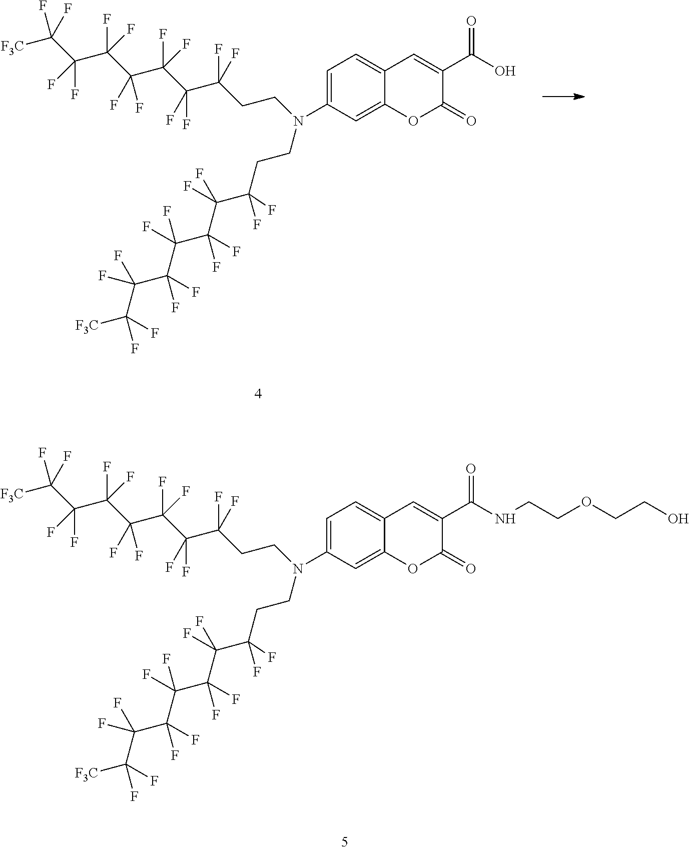 [SMILES: O=C(O)c1cc2ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc2oc1=O ; O=C(NCCOCCO)c1cc2ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc2oc1=O]

(5) Carboxylic Acid 4 (5.48 g, 5 mmol, 1 eq) is dissolved into a solution of 25 mL of dry DCM with anhydrous triethylamine (1.4 mL, 10 mmol, 2 eq) and EDCI-HCl (1.43 g, 7.5 mmol, 1.5 eq). Next a solution of 2-(2-aminoethoxy)ethanol (0.60 mL, 6 mmol, 1.2 eq) in 5 mL of anhydrous DCM is added dropwise and the reaction monitored by TLC for completion. The reaction is quenched with sat. NaHCO₃ (20 mL), washed with brine (20 mL), dried over Na₂SO₄ and concentrated in vacuo. The crude mixture is then purified via flash chromatography giving pure compound 5.

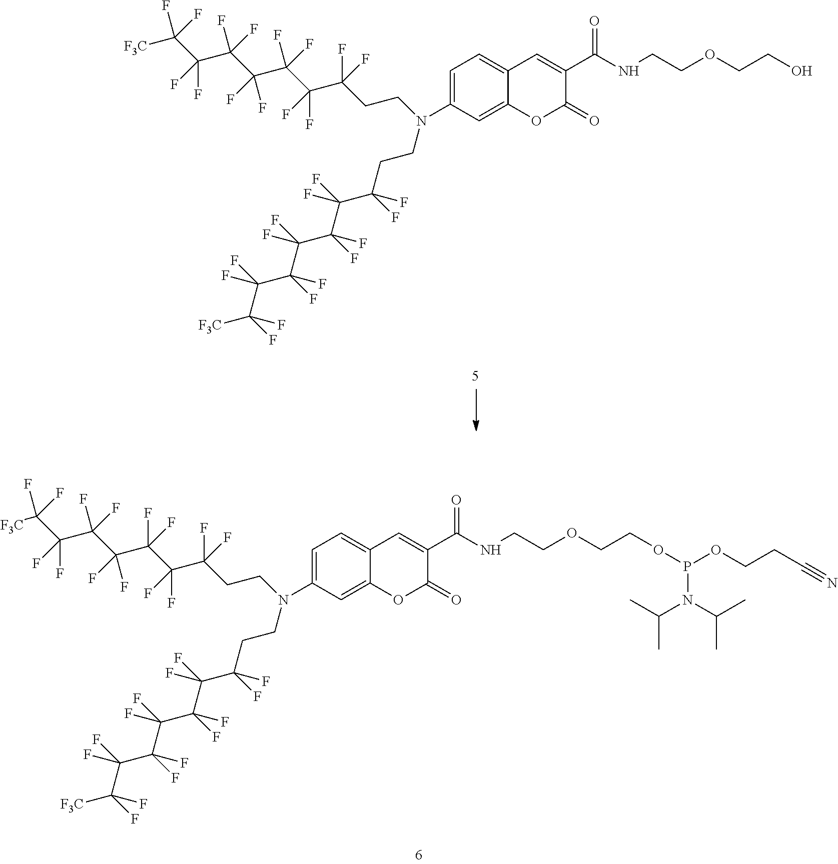 [SMILES: O=C(NCCOCCO)c1cc2ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc2oc1=O ; CC(C)N(C(C)C)P(OCCC#N)OCCOCCNC(=O)c1cc2ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc2oc1=O]

Coumarin-2FT-PA (6). Primary alcohol 5 (5.92 g, 5 mmol, 1 eq) is dissolved into 5 mL of anhydrous MeCN. DIPEA (1.8 mL, 10 mmol, 2 eq) is then added followed by the addition of 2-Cyanoethyl N,N-diisopropylchlorophosphoramidite (1.7 mL, 7.5 mmol, 1.2 eq). The reaction is let to sit at ambient temp for 12 hours before being concentrated down on a in vacuo. The crude product is then redissolved into 10 mL of toluene, azeotroped 3× and purified via flash chromatography giving FT-Coumarin Phosphoramidite 6 as a pure compound.

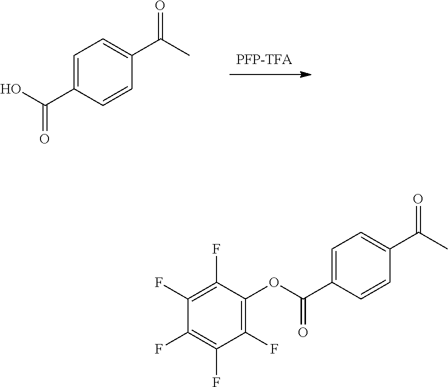 [SMILES: CC(=O)c1ccc(C(=O)O)cc1 ; CC(=O)c1ccc(C(=O)Oc2c(F)c(F)c(F)c(F)c2F)cc1]

4-acetyl-benzoic acid (3.28 g, 20 mmol, 1 eq) is added to a solution of anhydrous acetonitrile (100 mL) and triethylamine (5.57 mL, 40 mmol, 2 eq). RM is treated with pentafluorophenyltrifluoroacetate (5.60 g, 3.43 mL, 1 eq) dropwise and stirred for 2 hours at ambient temp. RM concentrated in vacuo until a solid begins to precipitate. The mixture is then dissolved into EtOAc (250 mL), washed with 10% citric acid (2×50 mL), sat. NaHCO₃ (2×100 mL), dried over Na₂SO₄ and concentrated in vacuo giving pure Acetylpentafluorophenyl benzoate (6.49 g, 98%) as a white crystalline solid. TLC in 10% EtOAc/Heptane Rf 0.5.

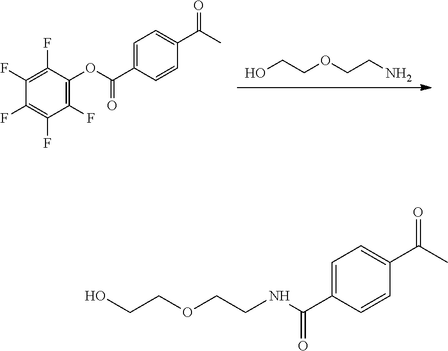 [SMILES: CC(=O)c1ccc(C(=O)Oc2c(F)c(F)c(F)c(F)c2F)cc1 ; NCCOCCO ; CC(=O)c1ccc(C(=O)NCCOCCO)cc1]

A solution of PFP-Ester (2.0 g, 2 mmol, 1 eq) in dry ACN (20 mL) is added dropwise to a stirred solution of 2-(2-aminoethoxy)ethanol (0.714 mL, 7.2 mmol, 1.2 eq) and DIEA (2.67 mL, 15 eq, 2.5 eq) in dry acetonitrile (20 mL). The reaction is stirred for 2 days at ambient temp and concentrated in vacuo. RM is then taken up in DCM and organic layer washed with sat. NaHCO₃, brine, dried over Na₂SO₄ and concentrated in vacuo giving the product (1.5 g, 100%) as a white solid which is taken directly into the next step without further purification. TLC in 10% MeOH/DCM Rf 0.25.

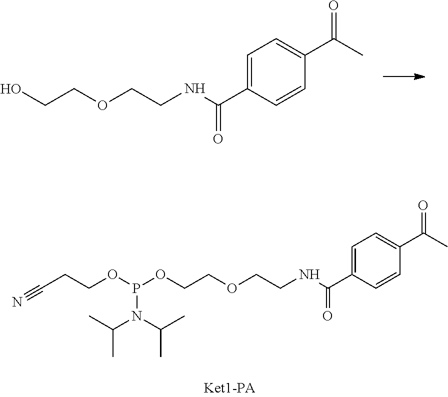 [SMILES: CC(=O)c1ccc(C(=O)NCCOCCO)cc1 ; CC(=O)c1ccc(C(=O)NCCOCCOP(OCCC#N)N(C(C)C)C(C)C)cc1]

To a solution of primary alcohol (1.5 g, 5.9 mmol, 1 eq) in anhydrous acetonitrile (12 mL) is added diisopropylethylamine (1.55 mL, 12 mmol, 2 eq) followed by dropwise addition of 2-Cyanoethyl N,N-diisopropylchlorophosphoramidite (1.97 mL, 8.85 mmol, 1.5 eq). The RM is allowed to sit at ambient temp for 3 hours and then concentrated in vacuo. RM is then dissolved into EtOAc, washed with sat. NaHCO₃, brine, dried over Na₂SO₄ and concentrated in vacuo giving Ket1-PA as a clear colorless oil.

### Synthesis of Ald-PA

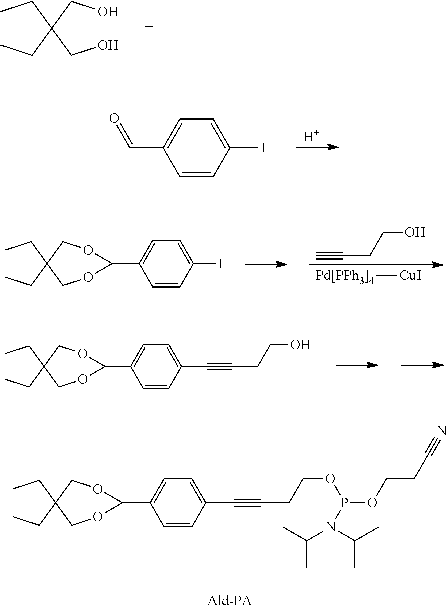

4-Iodobenzaldehyde (4.91 g, 21.2 mmol, 1 eq) is placed into a 500 mL round bottom and dissolved into toluene (100 mL) with 2,2-Diethyl-1,3-propanediol (3.17 g, 24 mmol, 1.2 eq) and camphorsulfonic acid (0.49 g, 2 mmol, 0.1 eq). The round bottom is then placed onto a rotovap with an 80° water bath, and the RM is azeotroped with 8×50 mL additions of toluene at which point water stops evolving from the reaction. The reaction mixture is then concentrated in vacuo and redissolved into EtOAc. The organic layer is washed with sat. NaHCO₃, brine, dried over Na₂SO₄ and concentrated in vacuo giving a white solid. Product is recrystallized from 15 mL of MeOH with 1 mL of H₂O added dropwise while heated to 70°. Slowly cooling to 0° gives the aryl iodide product (4.22 g, 60%) as white flakes. TLC 5% EtOAc/heptane Rf 0.61.

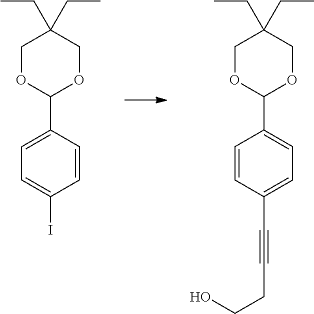 [SMILES: CCC1(CC)COC(c2ccc(I)cc2)OC1 ; CCC1(CC)COC(c2ccc(C#CCCO)cc2)OC1]

Copper iodide (0.327 g, 1.72 mmol, 0.2 eq), Pd(PPh₃)₄ (0.993 g, 0.86 mmol, 0.1 eq) and the aryl iodide (3.0 g, 8.6 mmol, 1 eq) are combined in flask with stir bar. The flask is placed on high vacuum overnight and the round bottom is refilled with argon. The solids are dissolved with DMF (17 mL) and Et₃N (2.39 mL, 17.2 mmol, 2 eq) is added. 3-Butyne-1-ol (0.779 mL, 10.3 mmol, 1.2 eq) is then added dropwise and the RM stirred at ambient temp for 3 hr. RM is then diluted with 60 mL of 0.1M Na-EDTA and 150 mL of EtOAc, stirred for 30 min and transferred to a separatory funnel with 300 mL of EtOAc. Organic layer washed with 0.1M Na₂EDTA, sat. NaHCO₃, brine, dried over Na₂SO₄ and concentrated in vacuo giving a dark brown/reddish oil. Purification via flash chromatograph gives the product (2.38 g, 80%) as a light-yellow solid. TLC 60%/6 EtOAc/heptane Rf 0.45.

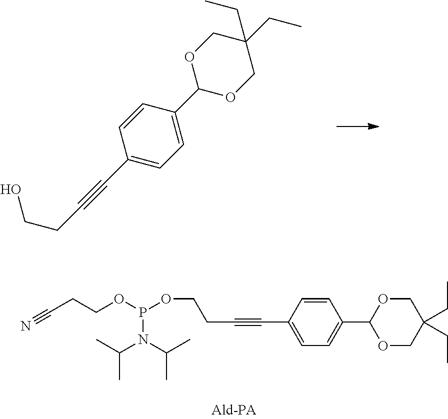 [SMILES: CCC1(CC)COC(c2ccc(C#CCCO)cc2)OC1 ; CCC1(CC)COC(c2ccc(C#CCCOP(OCCC#N)N(C(C)C)C(C)C)cc2)OC1]

Ta solution of primary alcohol (2.38 g, 8.3 mmol, 1 eq) in anhydrous acetonitrile (17 mL) is added diisopropylethylamine (3.0 mL, 12 mmol, 2 eq) followed by dropwise addition of 2-Cyanoethyl N,N-diisopropylchlorophosphoramidite (2.2 mL, 10 mmol, 1.2 eq). The RM is allowed to sit at ambient temp overnight. RM concentrated in vacuo, diluted with EtOAc, washed with sat. NaHCO₃, brine, dried over Na₂SO₄ and concentrated in vacuo giving Aid-PA as a clear yellow oil.

### Synthesis of Ket1-PA

The synthesis of the reagent is performed according to the following scheme:

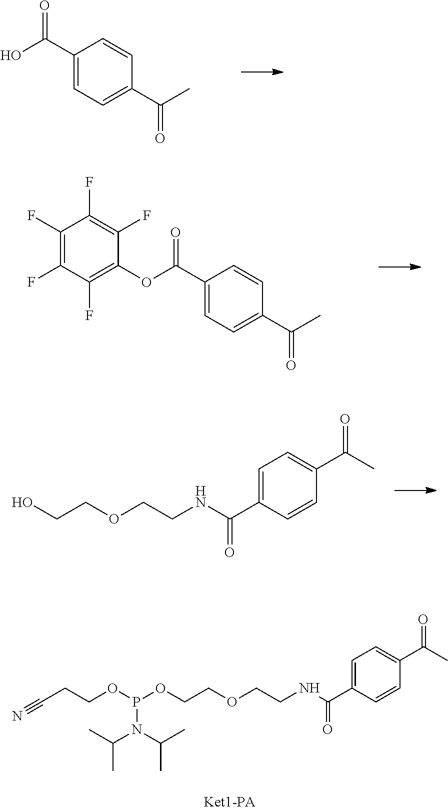 [SMILES: CC(=O)c1ccc(C(=O)O)cc1 ; CC(=O)c1ccc(C(=O)Oc2c(F)c(F)c(F)c(F)c2F)cc1 ; CC(=O)c1ccc(C(=O)NCCOCCO)cc1 ; CC(=O)c1ccc(C(=O)NCCOCCOP(OCCC#N)N(C(C)C)C(C)C)cc1]

### Synthesis of Ket2-PA

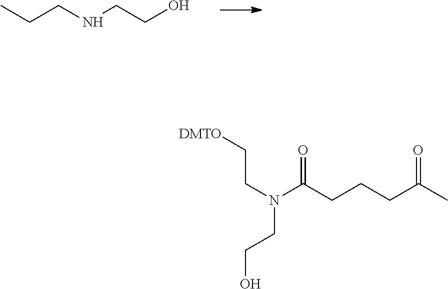

4-Acetylbutyric acid (5 g, 38 mmol, 1.5 eq) is dissolved into dry dichloromethane (100 mL) with triethylamine (7.0 mL 50 mmol, 1.3 eq). EDCI-HCl (26 mmol, 5 g, 1.1 eq) is added followed by dropwise addition of the amino alcohol (25 mmol, 10.2 g, 1 eq) as a solution in DCM/Et₃N (20:1, 50 mL). The reaction is stirred for 24 hours dissolved into 200 mL DCM and quenched with sat. NaHCO₃. The reaction is and washed with sat. NaHCO₃, brine, dried over Na₂SO₄ and concentrated in vacuo giving the crude product (17 g) as a yellow oil which is taken directly into the next step without further purification. TLC 5% Et₃N/10% EtOH/85% EtOAc Product Rf 0.55.

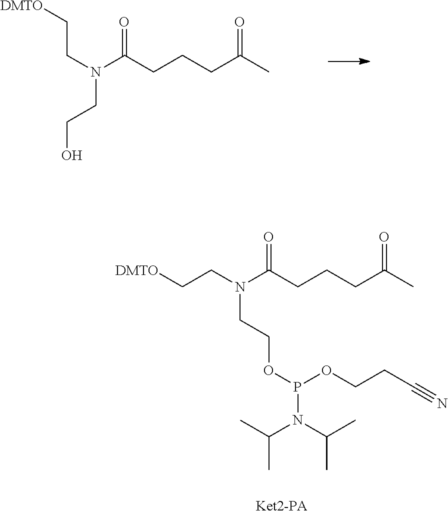

To a solution of keto-amide from the previous step (17 g impure) in anhydrous acetonitrile (38 mL) is added diisopropylethylamine (9.8 g, 13.5 mL, 76 mmol, 2 eq), followed by dropwise addition of 2-Cyanoethyl N,N-diisopropylchlorophosphoramidite (13.5 g, 57 mmol, 1.5 eq). The RM is allowed to sit at ambient temp for 3 hr, concentrated in vacuo, diluted with EtOAc, washed with sat. NaHCO₃, brine, dried over Na₂SO₄ and concentrated in vacuo. Purification via flash chromatograph provided the Phosphoramidite (4.97 g, 28% over 2 steps) as a viscous yellow oil. TLC 10% MeOH, 5% Et₃N, 85% EtOAc Rf 0.73.

### FT Hydrazides and Hydroxylamines

FT containing hydrazine and hydroxylamine reactive groups are synthesized according to the following scheme:

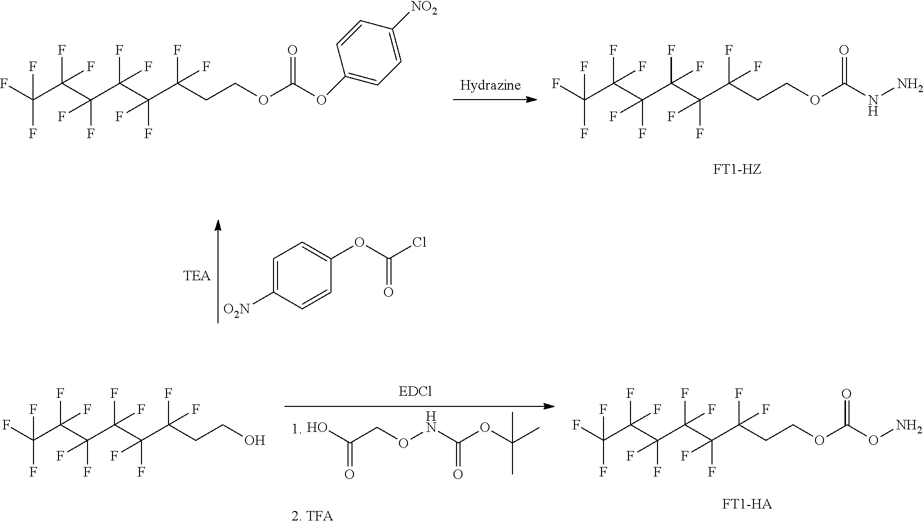

FT2-HZ and FT2-HA are synthesized by the same methods.

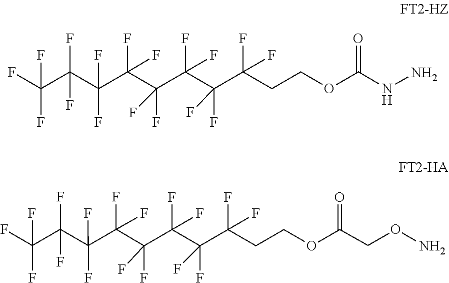 [SMILES: NNC(=O)OCCC(F)(F)C(F)(F)C(F)(F)C(F)(F)C(F)(F)C(F)(F)C(F)(F)C(F)(F)F ; CNOCC(=O)OCCC(F)(F)C(F)(F)C(F)(F)C(F)(F)C(F)(F)C(F)(F)C(F)(F)C(F)(F)F]

### Solubilizing Reagents SA1, SA2 and SA3

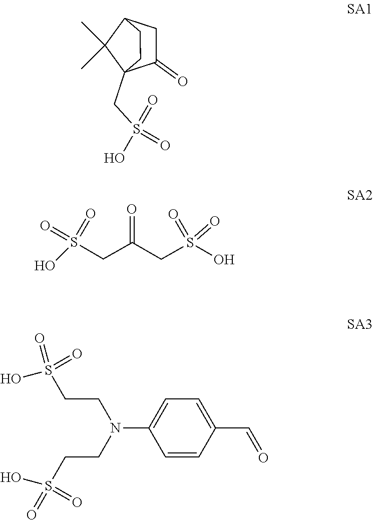 [SMILES: CC1(C)C2CCC1(CS(=O)(=O)O)C(=O)C2 ; O=C(CS(=O)(=O)O)CS(=O)(=O)O ; O=Cc1ccc(N(CCS(=O)(=O)O)CCS(=O)(=O)O)cc1]

10-Camphorsulfonic acid SA1 is commercially available (Sigma-Aldrich, cat. no. C2107).

Propanone-1,3-disulfonic acid SA2 is prepared by sulfonation of acetone with chlorosulfonic acid in methylene chloride according to Example 1, U.S. Pat. No. 5,430,180.

Bissulfonic aminobenzaldehyde SA3 is prepared according to WO2019213543 method for making bissulfonic aminobenzaldehyde:

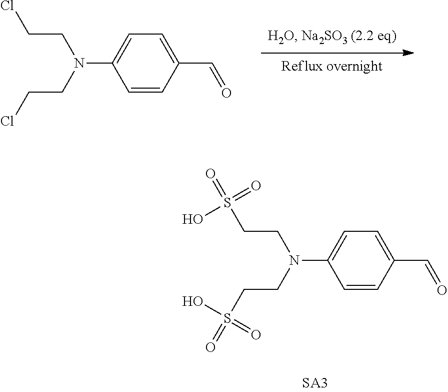 [SMILES: O=Cc1ccc(N(CCCl)CCCl)cc1 ; O=Cc1ccc(N(CCS(=O)(=O)O)CCS(=O)(=O)O)cc1]

General structure of water soluble FT hydrazides and oximes bound to sulfonic acid-containing aldehydes and ketones:

F(CF₂)ₙ(CH₂)₂—OC(O)(Gk)N=(Wm)(SO₃H)ₚ

General synthesis of such reagents can be represented by the following scheme:

F(CF2)ₙ(CH₂)₂—OC(O)(Gₖ)NH₂+O=(Wm)(SO₃H)_{p→}

→F(CF2)ₙ(CH₂)₂—OC(O)(Gₖ)N=(Wm)(SO₃H)ₚ+H₂O

In preferred embodiments n=6, 8; Gₖ are G₁=NH, or G₂=CH₂O; (Wm)(SO₃H)ₚ is a group formed by addition of sulfonic acid-containing aldehydes and ketones such as camphorsulfonic acid (SA1, p=1); 2-oxo-1,3-propanedisulfonic acid (SA2, p=2); and 2,2′-(4-formyl-phenylimino)-bis-ethanesulfonic acid (SA3, p=2).

### General Method of Preparation of Water Soluble FT Hydrazides and Oximes Bound to Sulfonic Acid-Containing Aldehydes and Ketones

FT compound (1 mmol) is dissolved in DCM (5 mL) and added to a stirred solution of SA compound (2 mmol) in MeOH (10 mL) and triethylamine (2 mmol, 0.28 mL). The combined solution is stirred at room temperature, and reaction is monitored by a RP TLC and KMnO₄ for development of spots on TLC plate. Reaction can be catalyzed by amines described in Chem. Sci., 2018, 9, 5252 (Dennis Larsen, Anna M. Kietrys, Spencer A. Clark, Hyun Shin Park, Andreas Ekebergh and Eric T. Kool. Exceptionally rapid oxime and hydrazone formation promoted by catalytic amine buffers with low toxicity). Once the reaction is finished, the reaction mixture is evaporated. Water added with subsequent addition of HCl that leads to precipitation of the product in the acid form. Neutralization with sodium or potassium hydroxide is leading to corresponding salts that are better soluble in water and oligo-compatible pH of the reagent in aqueous solutions.

Some examples of water soluble FT hydrazides FT1-HZ-SA2 and oximes FT1-HA-SA2 are obtained by reaction of corresponding FT hydrazide and oxime with SA2:

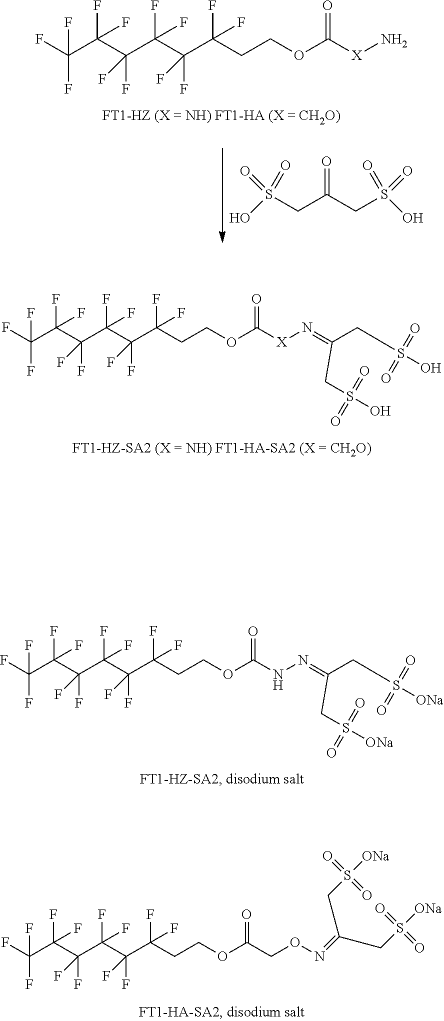

FT-modified oligonucleotides from ketone- and aldehyde-modified oligonucleotides by exchange reaction with water soluble FT hydrazides and oximes bound to sulfonic acid-containing aldehydes and ketones.

FT-modified oligonucleotides FT5-FT12 can be obtained by exchange reaction between corresponding ketone- and aldehyde-modified oligonucleotides by exchange reaction with water soluble FT hydrazides and oximes

### Synthesis of FT-Containing Oligos

Oligonucleotide probes with fluorinated tags (FT oligo) can be synthesized using ordinary phosphoramidite chemistry on oligonucleotide synthesizer. Shasta synthesizer (Sierra BioSystems, Inc., Sonora, CA) is used for making all oligonucleotides. Fluorinated tags are made using commercial reagents from Matrix Scientific and introduced to 5′-end of oligonucleotide probes forming FT by using corresponding phosphoramidites, azide, hydrazide, and hydroxylamine derivatives.

### FT HyP by Automated Oligo Synthesis Using FT Phosphoramidites

The automated oligo synthesis is a preferred method for building entire HyP on a synthesizer. As described above, FT can be introduced to either 3′- or 5′-end using corresponding phosphoramidites and commercially available Doublers and Treblers. Additionally, FT phosphoramidites pIA-2FTa pIA-2FTb, p-AA-2FT, pAPA6-2FT, pAPA8-2FT, pAB-2FT1 pIA-2FT, and Coumarin-2FT-PA all contain two FT in the reagents and therefore allow simultaneous introduction of two FT into HyP in a single coupling step. The following example illustrates the design of HyP with FT modification on 5′-end.

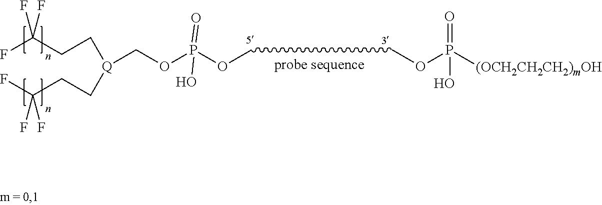

Q represents moieties incorporated into the probe from reagents pIA-2FTa pIA-2FTb, p-AA-2FT, pAPA6-2FT, pAPA8-2FT, pAB-2FT1 pIA-2FT, and Coumarin-2FT-PA.
FT HyP by Exchange of Ketone or Aldehyde-Containing Oligos with Water Soluble FT Hydrazides and Hydroxylamines

Oligos containing reactive aldehyde or ketone groups can be synthesized using reagents Ket1-PA, Ket2-PA, and Ald-PA. The latter has aldehyde protected in the phosphoramidite reagent, and after synthesis of oligo, it requires deprotection with 80% acetic acid at room temperature over two hours. Hydrazides and hydroxylamines are readily reactive with aldehydes and ketones, only if they are soluble under the reaction conditions. FT derivatized with hydrazides or hydroxylamine groups are not sufficiently soluble in water. To overcome this problem, the soluble adducts of these reagents are made by initial reaction with sulfonated ketones SA1, SA2 or SA3. The soluble forms of FT-HZ and FT-HA reagents can exchange with oligo aldehydes and ketones forming FT-containing HyP. Such exchange reaction can be performed with either individual probes or with the entire library of HyP making possible to delay introduction of FT into HyP if desired for enrichment step or for production process. The following is an illustration of such reaction where W represents moieties from reagents Ket1-PA, Ket2-PA, and Ald-PA.

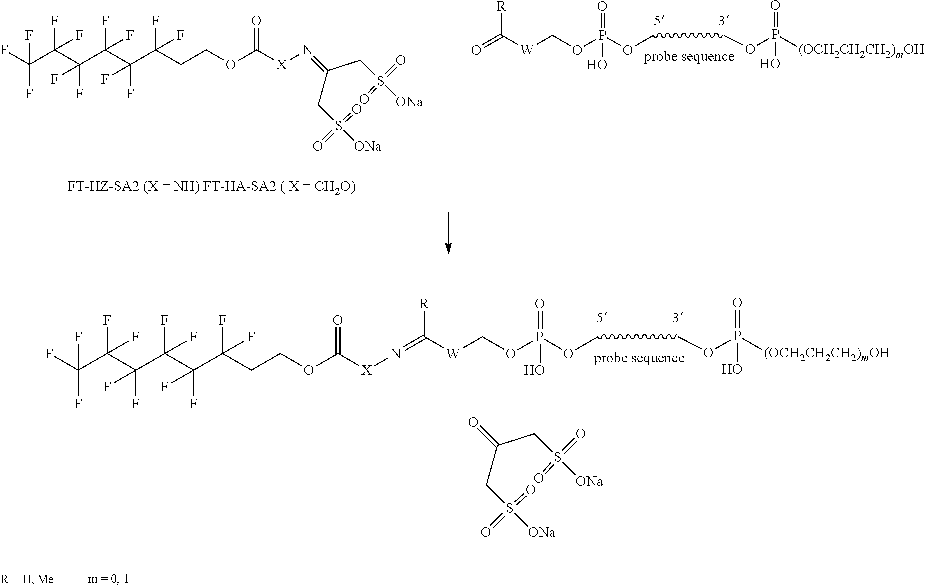

### FT HyP by Post-Synthetic Click-Coupling Using Alkyne-Containing Oligos and Azido-FT

Delayed introduction of FT into HyP can be achieved by an alternative method by utilizing Click chemistry. The advantage of this approach is in broad availability reagents and services for making alkyne-containing oligonucleotides. For example, the entire HyP library for enrichment with 5′-Hexynyl-modified oligos is available by ordering from Integrated DNA Technologies. Inc. Click chemistry is performed with Individual alkyne-containing HyP or with the entire library at once. FT derivatized with azide groups are not sufficiently soluble in water. To overcome this problem, the soluble forms of these reagents are made by one-step phosphoramidite coupling with Azido-CPG according to the following scheme.

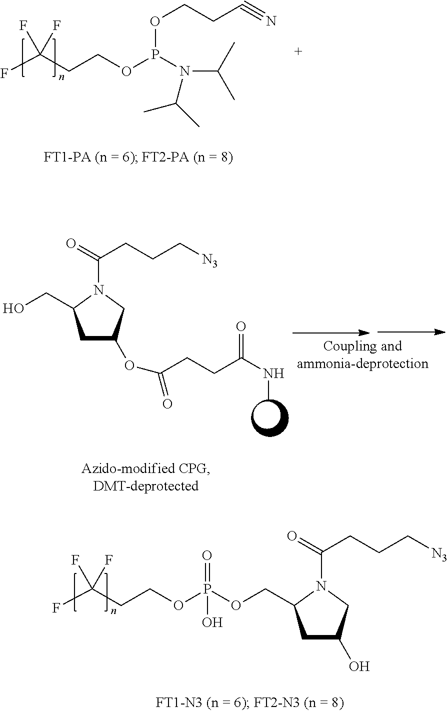

Subsequent Click conjugation is performed by using copper catalyst following standard protocol recommended by Lumiprobe (Click-Chemistry Labeling of Oligonucleotides and DNA). Excessive azide is scavanged by Alkyne Magnetic Beads commercially available from Click Chemistry Tools, Inc. (cat. no. 1035-1). The FT-conjugated oligos are purified on Fluoro-Pak™ column (#FP-7210) purchased from Berry&Associates.

### Hybridization Probes

Hybridization probes and their intermediates are designed as 80-mers with sequences complementary to expected targets. In one example, the synthesis is performed at a 50 nM scale using columns packed with 2000A Uni support from Biocomma Ltd. (China), cat. No. DS0050-2-3900. This method produces probes with free 3′-OH group. Another set of probes is synthesized on a 3′-Spacer C3 CPG1000 from AM Chemicals (Oceanside, CA) in a 0.2 mM scale. All the probes were terminated at the 5′-end with FT groups. Structures of FT groups are shown in Table 1. The designs are summarized in Table 2.

TABLE 1
STRUCTURES OF FT GROUPS.

| FT group | FT reagent | Structure of FT group |
|---|---|---|
| FT1 | | F(CF₂)₆(CH₂)₂— |
| FT2 | | F(CF₂)₈(CH₂)₂— |
| FT3 | | 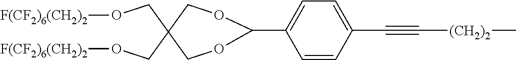 |
| FT4 | | 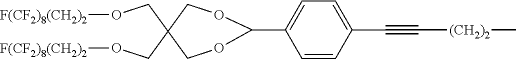 |
| FT5 | | 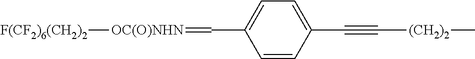 |
| FT6 | | 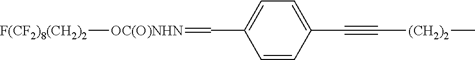 |
| FT7 | | 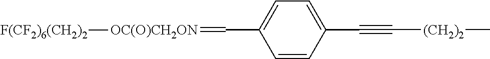 |
| FT8 | | 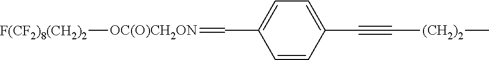 |
| FT9 | | 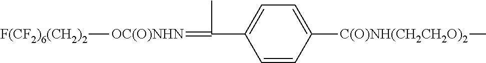 |
| FT10 | | 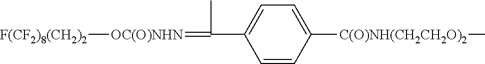 |
| FT11 | | 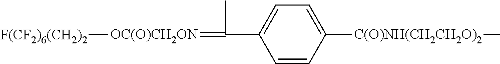 |
| FT12 | | 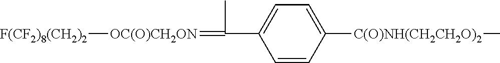 |

TABLE 2
HYBRIDIZATION PROBES AND THEIR INTERMEDIATES.

| Oligo | 5′ group | Linker | Oligo sequence | 3′ group |
|---|---|---|---|---|
| ODN1 | FT1 | —OP(O)(OH)— | Seq1 | —OH |
| ODN2 | FT1 | —OP(O(OH)— | Seq2 | —OH |
| ODN3 | FT1 | —OP(O(OH)— | Seq3 | —OH |
| ODN4 | FT1 | —OP(O(OH)— | Seq1 | —O(CH₂)₃OH |
| ODN5 | FT1 | —OP(O(OH)— | Seq2 | —O(CH₂)₃OH |
| ODN6 | FT1 | —OP(O(OH)— | Seq3 | —O(CH₂)₃OH |
| ODN7 | FT2 | —OP(O(OH)— | Seq1 | —OH |
| ODN8 | FT2 | —OP(O(OH)— | Seq2 | —OH |
| ODN9 | FT2 | —OP(O(OH)— | Seq3 | —OH |
| ODN10 | FT2 | —OP(O(OH)— | Seq1 | —O(CH₂)₃OH |
| ODN11 | FT2 | —OP(O(OH)— | Seq2 | —O(CH₂)₃OH |
| ODN12 | FT2 | —OP(O(OH)— | Seq3 | —O(CH₂)₃OH |
| ODN13 | FT1 | —OP(O)(OH)—(CH₂CH₂O)₆—P(O)(OH)— | Seq1 | —O(CH₂)₃OH |
| ODN14 | FT1 | —OP(O)(OH)—(CH₂CH₂O)₆—P(O)(OH)— | Seq2 | —O(CH₂)₃OH |
| ODN15 | FT1 | —OP(O)(OH)—(CH₂CH₂O)₆—P(O)(OH)— | Seq3 | —O(CH₂)₃OH |
| ODN16 | FT2 | —OP(O)(OH)—(CH₂CH₂O)₆—P(O)(OH)— | Seq1 | —O(CH₂)₃OH |
| ODN17 | FT2 | —OP(O)(OH)—(CH₂CH₂O)₆—P(O)(OH)— | Seq2 | —O(CH₂)₃OH |
| ODN18 | FT2 | —OP(O)(OH)—(CH₂CH₂O)₆—P(O)(OH)— | Seq3 | —O(CH₂)₃OH |
| ODN19 | FT3 | —OP(O)(OH)—(CH₂CH₂O)₆—P(O)(OH)— | Seq1 | —O(CH₂)₃OH |
| ODN20 | FT3 | —OP(O)(OH)—(CH₂CH₂O)₆—P(O)(OH)— | Seq2 | —O(CH₂)₃OH |
| ODN21 | FT3 | —OP(O)(OH)—(CH₂CH₂O)₆—P(O)(OH)— | Seq3 | —O(CH₂)₃OH |
| ODN22 | FT4 | —OP(O)(OH)—(CH₂CH₂O)₆—P(O)(OH)— | Seq1 | —O(CH₂)₃OH |
| ODN23 | FT4 | —OP(O)(OH)—(CH₂CH₂O)₆—P(O)(OH)— | Seq2 | —O(CH₂)₃OH |
| ODN24 | FT4 | —OP(O)(OH)—(CH₂CH₂O)₆—P(O)(OH)— | Seq3 | —O(CH₂)₃OH |
| ODN25 | FT5 | —OP(O(OH)— | Seq1 | —O(CH₂)₃OH |
| ODN26 | FT5 | —OP(O(OH)— | Seq2 | —O(CH₂)₃OH |
| ODN27 | FT5 | —OP(O(OH)— | Seq3 | —O(CH₂)₃OH |
| ODN28 | FT6 | —OP(O(OH)— | Seq1 | —O(CH₂)₃OH |
| ODN29 | FT6 | —OP(O(OH)— | Seq2 | —O(CH₂)₃OH |
| ODN30 | FT6 | —OP(O(OH)— | Seq3 | —O(CH₂)₃OH |
| ODN31 | FT7 | —OP(O)(OH)—(CH₂CH₂O)₆—P(O)(OH)— | Seq1 | —O(CH₂)₃OH |
| ODN32 | FT7 | —OP(O)(OH)—(CH₂CH₂O)₆—P(O)(OH)— | Seq2 | —O(CH₂)₃OH |
| ODN33 | FT7 | —OP(O)(OH)—(CH₂CH₂O)₆—P(O)(OH)— | Seq3 | —O(CH₂)₃OH |
| ODN34 | FT8 | —OP(O)(OH)—(CH₂CH₂O)₆—P(O)(OH)— | Seq1 | —O(CH₂)₃OH |
| ODN35 | FT8 | —OP(O)(OH)—(CH₂CH₂O)₆—P(O)(OH)— | Seq2 | —O(CH₂)₃OH |
| ODN36 | FT8 | —OP(O)(OH)—(CH₂CH₂O)₆—P(O)(OH)— | Seq3 | —O(CH₂)₃OH |
| ODN37 | FT7 | —OP(O(OH)— | Seq1 | —O(CH₂)₃OH |
| ODN38 | FT7 | —OP(O(OH)— | Seq2 | —O(CH₂)₃OH |
| ODN39 | FT7 | —OP(O(OH)— | Seq3 | —O(CH₂)₃OH |
| ODN40 | FT8 | —OP(O(OH)— | Seq1 | —O(CH₂)₃OH |
| ODN41 | FT8 | —OP(O(OH)— | Seq2 | —O(CH₂)₃OH |
| ODN42 | FT8 | —OP(O(OH)— | Seq3 | —O(CH₂)₃OH |
| ODN43 | FT9 | —OP(O(OH)— | Seq1 | —O(CH₂)₃OH |
| ODN44 | FT9 | —OP(O(OH)— | Seq2 | —O(CH₂)₃OH |
| ODN45 | FT9 | —OP(O(OH)— | Seq3 | —O(CH₂)₃OH |
| ODN46 | FT10 | —OP(O(OH)— | Seq1 | —O(CH₂)₃OH |
| ODN47 | FT10 | —OP(O(OH)— | Seq2 | —O(CH₂)₃OH |
| ODN48 | FT10 | —OP(O(OH)— | Seq3 | —O(CH₂)₃OH |
| ODN49 | FT11 | —OP(O(OH)— | Seq1 | —O(CH₂)₃OH |
| ODN50 | FT11 | —OP(O(OH)— | Seq2 | —O(CH₂)₃OH |
| ODN51 | FT11 | —OP(O(OH)— | Seq3 | —O(CH₂)₃OH |
| ODN52 | FT12 | —OP(O(OH)— | Seq1 | —O(CH₂)₃OH |
| ODN53 | FT12 | —OP(O(OH)— | Seq2 | —O(CH₂)₃OH |
| ODN54 | FT12 | —OP(O(OH)— | Seq3 | —O(CH₂)₃OH |
| ODN55 | FT3 | —OP(O(OH)— | Seq4 | —O(CH₂)₃OH |
| ODN56 | FT4 | —OP(O(OH)— | Seq4 | —O(CH₂)₃OH |

### Sequences of Nucleic Acids.

Unless otherwise indicated, all sequences are listed from 5′ to 3′-end.

Seq1
TTCATCCCGTCAACATTCAAACGGCCTGTCTCATCATGGAAGGCGCTGA
ATTTACGGAAAACATTATTAATGGCGTCGAG
Seq2
GTCGTGGCCTTGCTATTGACTCTACTGTAGACATTTTTACTTTTTATGT
CCCTCATCGTCACGTTTATGGTGAACAGTGG
Seq3
CCTGACCGTACCGAGGCTAACCCTAATGAGCTTAATCAAGATGATGCTC
GTTATGGTTTCCGTTGCTGCCATCTCAAAAA
Seq4
ATTATTATATATTATATATAAATATAATTAATTATTTTA

### Short Hybridization Probes (20-60-Mers).

Hybridization is performed at the same standard temperature as with using the long probes, at 58° except for shorter time 10-30 min.

### Materials and Conjugation Method

N-(2-hydroxyethyl)phenazinium chloride is synthesized according to S. G. Lokhov, et al., Bioconjugate Chem. 1992, 3, 5, 414-419). Oligos containing primary amino groups are conjugated to the reagent Phe using a protocol described in the paper.

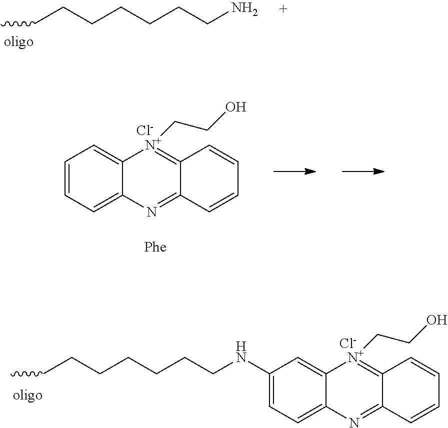

Amino linker can be introduced to the 3′-end using Glen Research reagents 3′-Amino-Modifier Serinol CPG (cat. no. 20-2997-14), and to the 5′-end with Amino-Modifier Serinol Phosphoramidite (cat. no. 10-1997-02). Symmetric Doubler Phosphoramidite (cat. no. 10-1920-02) is used to introduce two FT to the 5′-end of HyP. Y-Spacer C3 CPG from AM Chemicals LLC, 4065 Oceanside Blvd., Suite M Oceanside, CA 92056-5824 Fluoro-Pak™ cartridge (#FP-7210) for affinity purification.

### HyP Containing 3′-Terminal Phe Intercalating Group and Two 5′-FT.

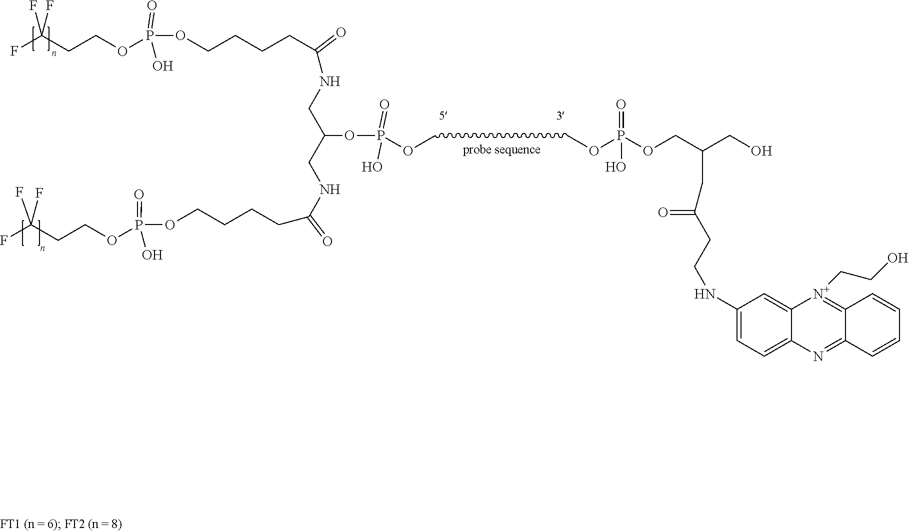

The probe is synthesized on oligo synthesizer by the following protocol.
1. Start synthesis using 3′-Amino-Modifier Serinol CPG
2. Build the probe sequence
3. Couple Symmetric Doubler Phosphoramidite following protocol recommended for the reagent by Glen Research
4. Couple with FT1-PA or FT2-PA
5. Deprotect from CPG with gaseous ammonia at 55° for three hr and elute with 0.5M NaCl to a fluorous cartridge
6. Wash the cartridge with water to remove failed sequences that do not contain FT
7. Wash with 5% acetonitrile in 0.1 M TEAA to remove the rest of the failures.
8. Elute purified HyP by 40% acetonitrile in 0.06 M TEAA buffer
9. Evaporate probes using Speedvac
10. For conjugation add 80 μL of 0.05 M N-(2-hydroxyethyl)phenazinium chloride (Phe) in 0.1 M aqueous Na₂CO₃ to the dried oligo and incubate at RT 10 min
11. Deposit the mixture with 0.5M NaCl to a fluorous cartridge and repeat cartridge purification following steps 6-8

### HyP Containing 5′-Terminal Phe Intercalating Group and Two 5′-FT.

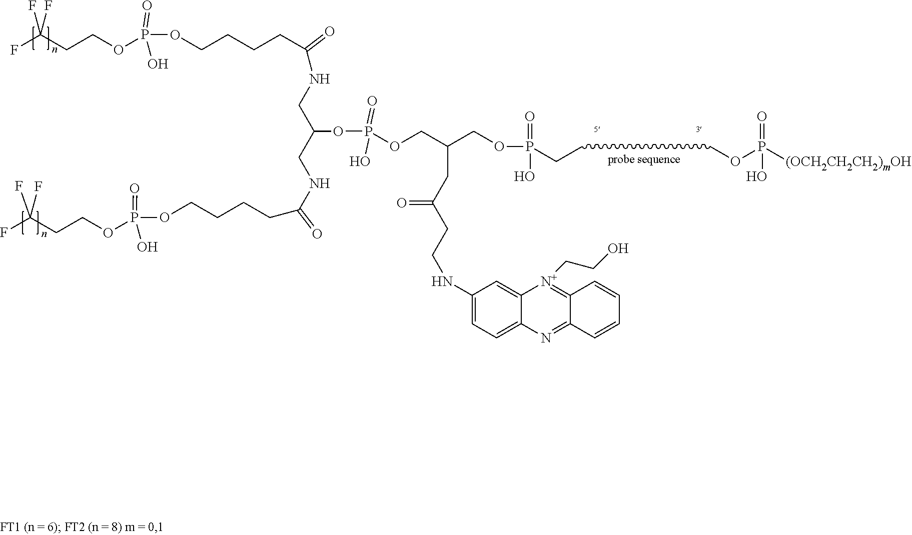

The probe is synthesized on oligo synthesizer by the following protocol.
1. Start synthesis using Universal CPG (for n=0) or 3-Spacer C3 CPG (for n=1)
2. Build the probe sequence
3. Couple Amino-Modifier Serinol Phosphoramidite following protocol recommended for the reagent by Glen Research
4. Couple Symmetric Doubler Phosphoramidite following protocol recommended for the reagent by Glen Research
5. Couple with FT1-PA or FT2-PA
6. Deprotect from CPG with gaseous ammonia at 55° for three hr and elute with 0.5M NaCl to a fluorous cartridge
7. Wash the cartridge with water to remove failed sequences that do not contain FT
8. Wash with 5% acetonitrile in 0.1 M TEAA to remove the rest of the failures.
9. Elute purified HyP by 40% acetonitrile in 0.06 M TEAA buffer
10. Evaporate probes using Speedvac
11. For conjugation add 80 μL of 0.05 M N-(2-hydroxyethyl)phenazinium chloride (Phe) in 0.1 M aqueous Na2CO3 to the dried oligo and incubate at RT 10 min
12. Deposit the mixture with 0.5M NaCl to a fluorous cartridge and repeat cartridge purification following steps 7-10

### HyP Containing Two 3′-, and 5′-Terminal Phe Intercalating Groups and Two 5′-FT.

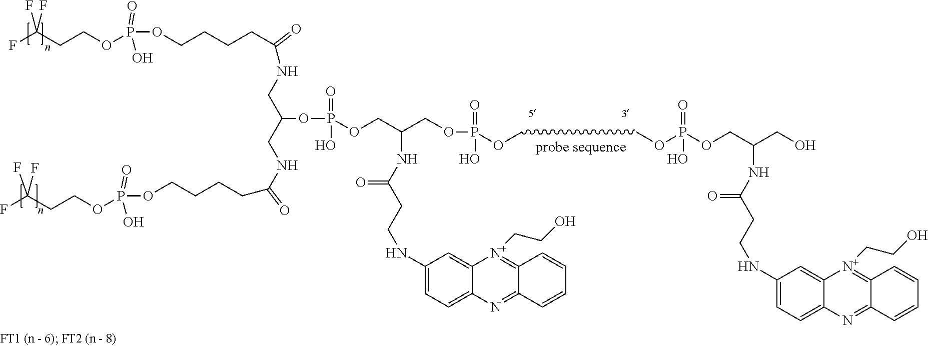

The probe is synthesized on oligo synthesizer by the following protocol.
1. Start synthesis using 3′-Amino-Modifier Serinol CPG
2. Build the probe sequence
3. Couple Amino-Modifier Serinol Phosphoramidite following protocol recommended for the reagent by Glen Research
4. Couple Symmetric Doubler Phosphoramidite following protocol recommended for the reagent by Glen Research
5. Couple with FT1-PA or FT2-PA
6. Deprotect from CPG with gaseous ammonia at 55° for three hr and elute with 0.5M NaCl to a fluorous cartridge
7. Wash the cartridge with water to remove failed sequences that do not contain FT
8. Wash with 5% acetonitrile in 0.1 M TEAA to remove the rest of the failures.
9. Elute purified HyP by 40% acetonitrile in 0.06 M TEAA buffer
10. Evaporate probes using Speedvac
11. For conjugation add 80 μL of 0.05 M N-(2-hydroxyethyl)phenazinium chloride (Phe) in 0.1 M aqueous Na₂CO₃ to the dried oligo and incubate at RT 10 min
12. Deposit the mixture with 0.5M NaCl to a fluorous cartridge and repeat cartridge purification following steps 7-10

### Evaluation of Enrichment by PCR Assay

The enrichment of fragmented DNA is evaluated by both the fold increase of the targeted region and the fold decrease of the untargeted region. A rough estimate of both is accomplished by qPCR with amplicons designed for either targeted or untargeted region. The change of CT in those qPCR reactions after enrichment is used as indications of the efficiency of enrichment reaction.

### Material

Pre-enrichment material: The fragmented DNA are Illumina short read sequencing libraries or simply fragmented genomic DNA of interest. For this specific example, human male DNA (Promega, Catalog #G1471), PhiX DNA (ThermoFisher, Catalog #SD0031), and T7 DNA (extracted in house from existing stocks) are made into Illumina sequencing library with part of Illumina DNA Prep with Enrichment, (S) Tagmentation (Illumina, Catalog #20025523). The pre-enriched pool is made of 100-200 ng of human DNA library and 0.75 fmol of each PhiX and T7 DNA libraries in each enrichment reaction (in 7.5 pL volume prior to enrichment), with additional volume of the same mixture for qPCR.

Enrichment reagent: Illumina RNA Fast Hyb Enrichment Beads+Buffers, and Illumina RNA Fast Hyb Enrichment PCR+Buffers, part of the Illumina RNA Prep with Enrichment, (L) Tagmentation kit (Illumina, Catalog #20040536).

Enrichment probes: Only T7 and PhiX phages are targeted in this experimental setting (sequences in the following table). The control probes are all single stranded DNA probe with a single biotinylation modification on its 5′ end/5Biosg/, ordered from IDT as individual 100 nmol DNA oligonucleotides with standard desalting. The received oligonucleotides were diluted into 125 pM each in the hybridization reaction. PhiX probes can be modified as experimental probes, while the T7 probe will stay the same as an internal control. Thermocycler QuantStudio3, channels FAM and VIC

| Target | Name | Sequence (5′-3′) |
|---|---|---|
| T7 | T2 | Seq5: |
| | | /5Biosg/GTTCTACCGTCCTGCACTCCTGTGATAATCCATTATGTTGTAACCCTGAACACCTATCCA |
| | | TAGGAACTCCAAAAGAGAAC |
| PhiX | P3 | Seq6: |
| | | /5Biosg/TTCATCCCGTCAACATTCAAACGGCCTGTCTCATCATGGAAGGCGCTGAATTTACGGAA |
| | | AACATTATTAATGGCGTCGAG |
| | P4 | Seq7: |
| | | /5Biosg/GTCGTGGCCTTGCTATTGACTCTACTGTAGACATTTTTACTTTTTATGTCCCTCATCGTCA |
| | | CGTTTATGGTGAACAGTGG |
| | P5 | Seq8: |
| | | /5Biosg/CCTGACCGTACCGAGGCTAACCCTAATGAGCTTAATCAAGATGATGCTCGTTATGGTTT |
| | | CCGTTGCTGCCATCTCAAAAA |

Where /5Biosg/ is a tag introduced using u-Biotin Phosphoramidite.

qPCR reagents: Master mix PERFECT MULTI QPCR TOUGH LOW ROX 250R (5× master mix, QuantaBio distributed through VWR under Catalog #89497-294). Human MT-ATP6 (Hs02596862_g1) qPCR 20× assay master mix is purchased from ThermoFisher (Catalog #4351370), serving as representative of untargeted host DNA. qPCR primer/probe sequences are shown in the following table. Primers are diluted to a 20× stock concentration of 8 μM each, and the probe are 4 M each in the same 20× stock.

| Amplicon | Oligo | | | | |
|---|---|---|---|---|---|
| name | name | Region | 5′-dye | sequence (5′-3′) | 3′-dye |
| PhiX16 | PhiX16-F | untarget | none | Seq 9: | none |
| | | | | CTCAAACGGCTGGTCAGTAT | |
| | PhiX16- | untarget | YY | Seq 10: | BHQ-2 |
| | probe | | | ACTCGCAAGGTTAGTGCTGAGGTT | |
| | PhiX16-R | untarget | none | Seq 11: | none |
| | | | | CTGATTCTGCGTTTGCTGATG | |
| PhiX17 | PhiX17-F | untarget | none | Seq 12: | none |
| | | | | TTTAGAGAACGAGAAGACGGTTA | |
| | PhiX17- | untarget | YY | Seq 13: | BHQ-2 |
| | probe | | | TGGCTGCTGAACGCCCTCTTAA | |
| | PhiX17-R | untarget | none | Seq 14: | none |
| | | | | GGGTAATTATACTCATCGCGAATATC | |
| PhiX3 | PhiX3-F | target | none | Seq 15: | none |
| | | | | GCTGCCGTCATTGCTTATTATG | |
| | PhiX3- | target | FAM | Seq 16: | BHQ-1 |
| | probe | | | CATCCCGTCAACATTCAAACGGCC | |
| | PhiX3-R | target | none | Seq 17: | none |
| | | | | TCCGTAAATTCAGCGCCTT | |
| PhiX4 | PhiX4-F | target | none | Seq 18: | none |
| | | | | TCTCCGTCTTTCTCCATTGC | |
| | PhiX4- | target | FAM | Seq 19: | BHQ-1 |
| | probe | | | CGTGGCCTTGCTATTGACTCTACTGT | |
| | PhiX4-R | target | none | Seq 20: | none |
| | | | | CGTGACGATGAGGGACATAAA | |
| PhiX5 | PhiX5-F | target | none | Seq 21: | none |
| | | | | CGTGACGATGAGGGACATAAA | |
| | | | | TTTAAAGCGCCGTGGATG | |
| | PhiX5- | target | FAM | Seq 22: | BHQ-1 |
| | probe | | | TGACCGTACCGAGGCTAACCCTAA | |
| | PhiX5-R | target | none | Seq 23: | none |
| | | | | GCAGCAACGGAAACCATAAC | |
| T7-4 | T7-4-F | target | none | Seq 24: | none |
| | | | | TCATCGCGTAATGTCTAATGCTCCGAA | |
| | | | | AG | |
| | T7-4-probe | target | /56FAM/ | Seq 25: | /3IABKFQ/ |
| | | | | CCCTGAACA/ZEN/CCTATCCATAGGAA | |
| | | | | CTCCA | |
| | T7-4-R | target | none | Seq 26: | none |
| | | | | GCGACCCTTATTTACCATGTCAGTGGA | |
| T7 | T7-F | untarget | none | Seq 27: | none |
| | | | | GGTCCAAGCTATTGGGTCTATC | |
| | T7-probe | untarget | /56FAM/ | Seq 28: | /3IABKFQ/ |
| | | | | AGCGGCTAT/ZEN/CGGAGAGAGTATGC | |
| | | | | T | |
| | T7-R | untarget | none | Seq 29: | none |
| | | | | GTCCTTCTGTGACTCGCTTAAT | |

YY is Yakima Yellow dye (#10-5920-95); BHQ-1 (#20-5931-42A) and BHQ-2 (#20-5932-42A) quenchers are from Glen Research. FAM dye (#F5160) from Lumiprobe. T7-4 and T7 probes with corresponding dyes coded are ordered from IDT.

### Methods:

### Hybridization

1. Add the following reagents in the order listed to a new strip tube.
   i. Pre-enriched library (7.5 μL)
   ii. Hyb Buffer 2+IDT NXT Blockers (NHB2)(12.5 μL) vortexed and heated to 50° C. prior to adding to the reaction
   iii. Enrichment probe panel (2.5 μL) thawed and vortexed prior to adding to the reaction
   iv. Enrich Hyb Buffer 2 (EHB2) (2.5 μL) thawed and vortexed prior to adding to the reaction
2. Pipette to mix, quick spin
3. Incubate the samples according to the following program on a thermocycler

| Step | Temp (° C.) | Time | Cycle # |
|---|---|---|---|
| 1 | 95 | 5 min | 1 |
| 2 | 94 | 1 min | 1 |
| 3 | 92 | 1 min | 1 |
| 4 | 90 | 1 min | 1 |
| 5 | 88 | 1 min | 1 |
| 6 | 86 | 1 min | 1 |
| 7 | 84 | 1 min | 1 |
| 8 | 82 | 1 min | 1 |
| 9 | 80 | 1 min | 1 |
| 10 | 78 | 1 min | 1 |
| 11 | 76 | 1 min | 1 |
| 12 | 74 | 1 min | 1 |
| 13 | 72 | 1 min | 1 |
| 14 | 70 | 1 min | 1 |
| 15 | 68 | 1 min | 1 |
| 16 | 66 | 1 min | 1 |
| 17 | 64 | 1 min | 1 |
| 18 | 62 | 1 min | 1 |
| 19 | 60 | 1 min | 1 |
| 20 | 58 | 90 min | 1 |

Total time: ˜120 minutes. Cover temperature: 100°.

Testing retention of fluorine-tagged hybridized probes and elution of a target on fluorinated column by chromatography

### Method of Capturing of FT HyP and Elution of a Target on Fluorinated Affinity Column by Chromatography

The retention of fluorine-tagged hybridized probes on a fluorinated column and elution of the target is performed by high-pressure liquid chromatography (HPLC) with diode-array detection (DAD). While enriched samples may not be detectable by DAD, the elution parameters (buffers, gradients, and temperature) are established by retention and elution of synthetic complementary target with one of the probes. This can be accomplished by monitoring UV spectra in real time for higher concentrations. The same elution parameters are used to retain real targeted nucleic acid that are hybridized with FT HyP, wash all untargeted nucleic acids and then elute the hybridized nucleic acid by change of the gradient, increase in temperature or both. The eluted and collected targeted nucleic acid can be used in consequent qPCR assay after desalting and concentrating.

### Materials

1. Varian HPLC system with DAD (or similar)
2. Phenomenex Luna 5 um PFP(2) 100A LC column 250×4.6 mm and corresponding guard column
3. DI water
4. OmniSolv ACN for HPLC

### Protocol

1. Turn on the column heater to a desired temperature up to 95° C. Equilibrate HPLC column with starting mobile phase (ACN:H2O=3:97) for at least 15 minutes.
2. Inject hybridized probe and make sure it is retained on the column. All non-targeted DNA fragments are not retained and eluted on this step, and that fraction is discarded.
3. Increase the column temperature to 95° C. to melt the target DNA from the probe. Collect the fraction with the target DNA and place on speedvac to remove the solvent. This sample is used in qPCR assay.
4. Increase ACN percentage in mobile phase to 90% to elute retained HyP and recover the column before the next injection.

### Method of Capturing of FT HyP and Elution of a Target Using Solid-Phase Extraction Materials

### Materials.

Columns for fluorous affinity enrichment: empty microcentrifuge spin columns with polyethylene frits (Biocomma, 007400) are packed with 75-200 mg of polytetrafluoroethylene (PTFE) powder (Goodfellow Cambridge, LS548583) or fluorinated silica gel (Fluka Analytical, 40915).

### Method.

In brief, a column is packed with a fluorinated sorbent. A pre-enriched library is hybridized with probes that have fluorinated tails. This library is passed through the column, where the fluorinated probes and their sequence-specific targets are retained preferentially over other DNA. The column is then washed under conditions which remove preferentially any non-targeted DNA without a hybridized fluorous probe. The targeted DNA is then eluted off the column under denaturing conditions, which might include chemical (high pH, such as in 200 mM NaOH solution) or temperature (raising the column above the melting temperature of the duplex probe-target DNA) and collected, where it may be desalted and concentrated for subsequent analysis.

### Preparing the Column, Loading DNA, Washing, and Elution of Targeted DNA

1. 75-200 mg of fluorinated sorbent is weighed and packed between two polyethylene frits in a Biocomma 7400 spin column.
2. Either positive air pressure or a microcentrifuge (e.g., an Eppendorf MiniSpin) is used to pass the following solutions in 600 μL volumes to prepare the column: acetonitrile, 0.1 M triethylammonium acetate, and 100 mg/mL NaCl with 5% (w/v) dimethylformamide (DMF)
3. Pre-enriched library hybridized with fluorous-tagged probes is diluted into 300 μL of 100 mg/mL NaCl with 5% DMF and passed through column over 30 to 90 seconds. DNA not bound to fluorous probes predominantly passes through the column and is discarded.
4. 1 mL of 10% acetonitrile (v/v) 0.1 M TEAA is passed through the column to remove remainder of DNA not bound to fluorous probes and discarded.
5. 300 μL of 200 mM NaOH in 100 mg/mL NaCl with 5% DMF is passed through the column to elute targeted DNA from hybridization probes
6. 48 μL of 1.25 M Tris pH 7.4 is added to the elution to lower the pH
7. The collected elution is desalted and concentrated in 25 μL ultra pure water with a centrifugal filter unit (e.g., MilliporeSigma UFC500396) 8. The enriched library is then amplified and characterized by qPCR

A variation on the above protocol is used to increase loading capacity and strength of interaction by adding a fluorinated liquid phase associated with the fluorinated solid support, allowing for more effective separation between fluorous tagged species and untagged DNA: after packing the column and rinsing with acetonitrile, 1% (v/v) perfluorodecalin (PFD) in acetonitrile is run through the column, followed by 50 μL neat PFD, and finally 600 μL ultra pure water. The sample is then loaded directly from water without the 100 mg/mL NaCl 5% DMF loading buffer. The sample is then able to retain without any counterions present (e.g., TEA or Na⁺) for subsequent washing and elution steps. The column is washed with water or 10% acetonitrile to remove background DNA and the targeted DNA and fluorous hybridization probes are eluted with 30% acetonitrile. Alternatively, the hybridized targets are eluted by heating the column to 95° C. for 5 minutes and washing with 300 μL of water heated to 95° C. The sample is concentrated as described above, amplified, and characterized by qPCR.

### Method of Capturing of FT HyP and Elution of a Target Using a Combination of Fluorous Liquids and Solid-Phase Extraction Materials

### Materials.

Perfluorodecalin (PFD, Aldrich P9900), ethanol, phosphate buffered saline (PBS, Sigma-Aldrich, 806552)

### Method.

PFD (50 μL) is added to 300 μL of 50% ethanol (v/v) PBS. Pre-enriched library hybridized to fluorous-tagged probes is added to the tube and agitated vigorously for 15 minutes on a heated shaker at 58° C., then spun down at 10,000 rpm for 2 minutes. 250 μL of supernatant is removed and 250 μL of 50% ethanol PBS is added back. These steps are repeated 3x for a total of four washes. On the fourth step, 15 μL of 2 N NaOH is added, and the solution is agitated at 58° C. for 2 minutes to elute targeted DNA into the aqueous phase. The tube is again spun down at 10,000 rpm for 2 minutes. 300 μL of supernatant is removed and placed on a centrifugal filter unit with a suitable molecular weight cutoff filter (e.g., 3 kDa or 10 kDa), where it is desalted and concentrated into 25 μL ultra pure water for subsequent amplification and characterization by qPCR.

### Method of Capturing of Hybridization Probes Containing FT and Biotin Label

In some embodiments the HyP contains both FT and biotin label. Such probes can be used in methods based on biotin affinity capturing with streptavidin surfaces and with fluorous surfaces. Such HyP can be synthesized using Asymmetric Doubler placed on the 5′-end and subsequent introduction of FT and Biotin labels to the 5′-end of the HyP.

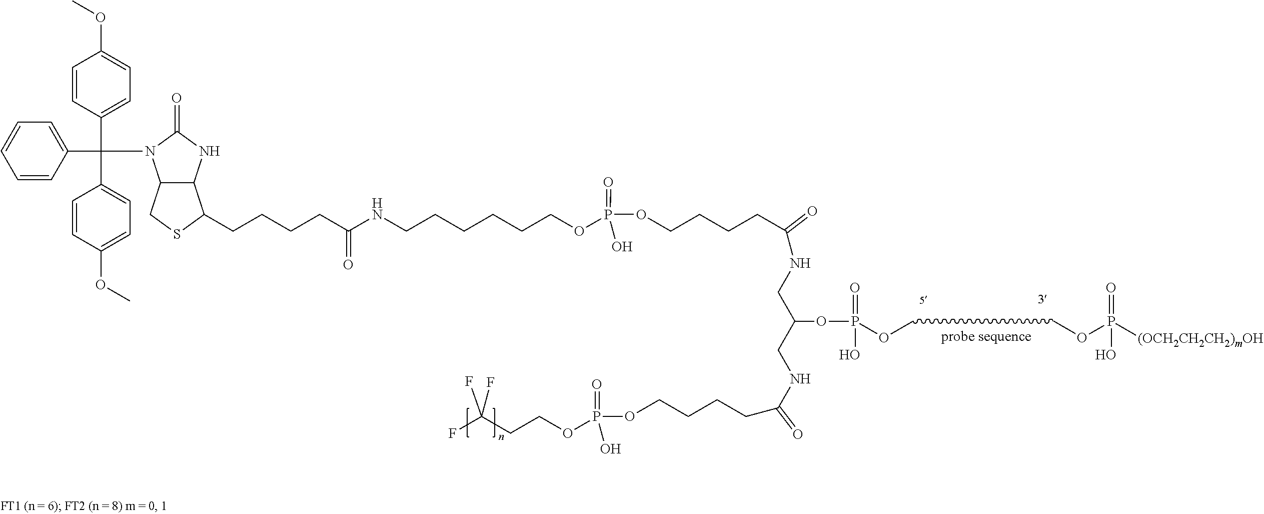

Alternatively, FT and biotin label containing HyP can be synthesized by using Biotin phosphoramidite (hydroxyprolinol) reagent (Lumiprobe Corporation, cat. no. 42360) that allows internal incorporation during automated synthesis

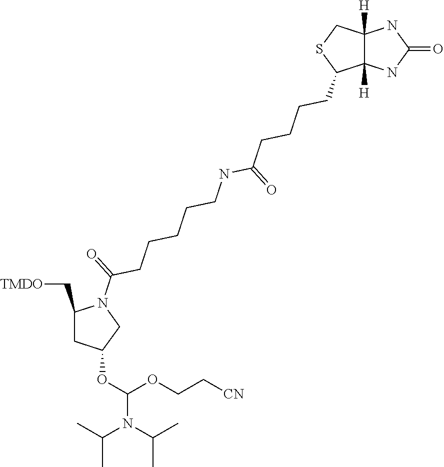

### Biotin Phosphoramidite (Hydraxyprolinol)

The HyP have the following structure:

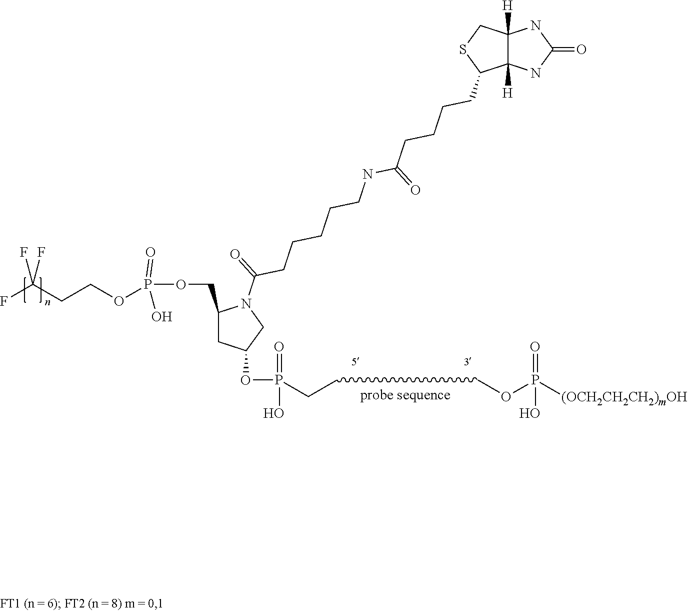

The following example is illustrating the use of such hybrid FT and biotin-containing HyP for enrichment with subsequent capturing using Streptavidin magnetic beads.

### Protocol

1. Add 62.5 μL Streptavidin Magnetic Beads (SMB) (vortexed after reaching room temperature for 30 minutes) to each sample, pipette slowly to fully resuspend beads.
2. Incubate in the thermocycler at 58° for 15 min.
3. Immediately after the above incubation, quick spin the tube strip and place it on a magnetic stand
4. When the liquid is clear, aspirate and discard the supernatant, remove the tube from the magnetic stand.
5. Add 50 μL preheated Enhanced Enrichment Wash (EEW)(58°), vortex the tube to resuspend the beads.
6. Place the strip tube on a thermocycler, close the lid, and incubate for five minutes at 58° C.
7. Remove the tube from the heat block, quick spin
8. Immediately place on a magnetic stand.
9. When the liquid is clear, aspirate and discard the supernatant, remove the tube from the magnetic stand.
10. Repeat steps 5-9 two times for a total of three washes.
11. After the third wash, add 50 μL preheated in EEW to the beads, vortex to resuspend.
12. Transfer 50 μL resuspended beads to a new PCR strip tube, vortex to resuspend.
13. Place the strip tube on a thermocycler, close the lid, and incubate for five minutes at 58°.
14. Remove the tube from the heat block, quick spin.
15. Immediately place on a magnetic stand and wait until the liquid is clear.
16. Using a pipette set to 50 μL, remove and discard all supernatant from each tube.
17. Quick spin the tube strip, place it back on the magnetic stand.
18. Use a P20 pipettor to remove any residual liquid from each sample.
19. Add 23 μL Elution Master Mix (table below) to each tube, and then vortex at high speed three times for 10 seconds each.

| Reagent | Per reaction (μL) |
|---|---|
| Enrichment Elution Buffer 1 (EE1) | 28.5 |
| 2 N NaOH (HP3) | 1.5 |
| Total | 30 |

1. Incubate the tube at room temperature for two minutes.
2. Quick spin the tube strip and place it on a magnetic stand and wait until the liquid is clear.
3. Transfer 21 μL supernatant to a new strip tube.
4. Add 4 μL Elute Target Buffer 2 (ET2), pipette to mix.
5. Quick spin.
6. Amplification of the Enriched Libraries
1. Thaw Enhanced PCR Mix (EPM) and PCR Primer Cocktail (PPC) on ice, Invert to mix, then centrifuge briefly.
2. Allow AMPure XP beads and Resuspension Buffer (RSB) to stand at room temperature for 30 minutes prior to use.
3. Add 5 μL of PPC in each tube.
4. Add 20 μL EPM to each tube and mix by pipetting.
5. Incubate the samples according to the following program on a thermocycler.

| Step | Temp (° C.) | Time | Cycle # |
|---|---|---|---|
| 1: Initial denaturation | 98 | 30 sec | 1 |
| 2: Denaturation | 98 | 10 sec | 14 |
| 3: Primer annealing | 60 | 30 sec | |
| 4: Extension | 72 | 30 sec | |
| 5: Final extension | 72 | 5 min | 1 |
| 6: Final hold | 10 | ∞ | 1 |

Total time: ˜35 minutes. Cover temperature: 100°.
1. Remove the tube from the thermocycler, quick spin.
2. Vortex and invert AMPure XP Beads, add 90 μL AMPure XP Beads to each tube, vortex on highest speed three times for 10 seconds each.
3. Incubate the tube at room temperature for 5 min.
4. Prepare fresh 80% ethanol.
5. After the incubation, quick spin and place on a magnetic stand.
6. When the liquid is clear, aspirate and discard all supernatant from the tube.
7. Add 175 μL of 80% ethanol without disturbing the beads.
8. Let the strip tubes stand for 30 seconds, remove and discard the supernatant.
9. Repeat steps 12-13 for a total of two washes.
10. Spin the tube and aspirate all the residual ethanol with a P20 pipette.
11. Leave tube strip on magnetic stand and air-dry for 2 min.
12. Remove from the magnetic stand, add 31 μL of RSB to each tube, pipette to mix and vortex for 5 sec.
13. Incubate samples at room temperature for 5 min, quick spin.
14. Place the tube strip on the magnetic stand.
15. When the liquid is clear, transfer 30 μL of enriched library into a new tube strip: this is the enriched library.
   qPCR and Data Analysis

For each condition: pre-enriched library, enriched library, blank (ddH2O only), make the following master mix for 18 reactions:
72 μL 5×qPCR reagent
265 μL ddH2O
5 μL Template (pre-enriched library mix, enriched library mix, or ddH2O)

Aliquot 19 μL of the above master mix into each reaction well of the qPCR plate, total 16 reactions.

In duplicate for each master mix, add 1 μL of the 20× primer/probe mix for each of the qPCR reaction. In the current example, each primer/probe mix has 6 reactions.

On the qPCR instrument, run the following program:

| Step | Temp (° C.) | Time | Cycle # |
|---|---|---|---|
| 1: Initial denaturation | 95 | 5 min | 1 |
| 2: Denaturation | 95 | 15 sec | 40 |
| 3: Primer annealing | 60 | 30 sec | |
| | | (detection) | |

Use Rox as passive reference, detect VIC channel for PhiX16 and PhiX17 qPCR reactions, FAM channel for all other reactions.

No amplification should happen in any reaction for the blank control set.

For the pre-enriched and enriched libraries, the CT are analyzed in the following groups

### PhiX

| | | Pre- | | | |
|---|---|---|---|---|---|
| | | enriched | Enriched | Change after | Final |
| amplicon | region | library | library | enrichment | evaluation |
| PhiX3 | target | Average | Average | CT1-CT2 = | ΔCT_{target} + |
| PhiX4 | target | CT1 | CT2 | ΔCT_{target} | ΔCT_{untarget} |
| PhiX5 | target | | | | ΔΔCT_{PhiX} |
| PhiX16 | untarget | Average | Average | CT4-CT3 = | |
| PhiX17 | untarget | CT3 | CT4 | ΔCT_{untarget} | |

The higher ΔΔCT_{PhiX}, the higher the enrichment efficiency for the PhiX genome T7

The higher ΔΔCT_{T7}, the higher the enrichment efficiency for the T7 genome. If the T7 library and probe are consistent across different experiments, this can be used as an internal control of enrichment efficiency.

### Human MT-ATP6

| | | Pre- | | | |
|---|---|---|---|---|---|
| | | enriched | Enriched | Change after | Final |
| amplicon | region | library | library | enrichment | evaluation |
| T7-4 | target | Average | Average | CT1-CT2 = | ΔCT_{target} + |
| | | CT1 | CT2 | ΔCT_{target} | ΔCT_{untarget} = |
| T7 | untarget | Average | Average | CT4-CT3 = | ΔΔCT_{T7} |
| | | CT3 | CT4 | ΔCT_{untarget} | |
| | | Pre- | | | |

| | | enriched | Enriched | Change after |
|---|---|---|---|---|
| amplicon | region | library | library | enrichment |
| MT-ATP6 | untarget | Average | Average | CT3-CT4 = |
| | | CT1 | CT4 | ΔCTₕᵤₘₐₙ |

Here the ΔCTₕᵤₘₐₙ should be a negative value, representing the level of reduction of host content after the enrichment.

The above data can be graphed as shown in FIG. 7, the phiX and T7 bars represent relative enrichment level among the experiments and the Human bar represent the depletion of the host signal after enrichment.

1. A hybridization probe comprising a) a polynucleotide having a 3′ end and a 5′ end and comprising about 20 to about 200 nucleotide units and b) one or more fluorinated affinity tags, wherein each affinity tag comprises one or more polyfluorinated carbon chains each comprising 3-30 carbon atoms; wherein the polynucleotide comprises a sequence complementary or substantially complementary to a target sequence within a target nucleic acid.

2. The hybridization probe of claim 1, wherein the hybridization probe has a structure:
[(FT)ₙ-Y-L]ₘ-HyS
wherein:
FT is a fluorinated carbon affinity tag comprising one or more polyfluorinated carbon chains each comprising 3-30 carbon atoms;
n=is 1, 2, or 3;
m=1 or 2;
HyS is a polynucleotide having a 3′ end and a 5′ end and comprising about 20 to about 200 nucleotide units;
L is an optional linker moiety connecting HyS and Y, wherein L is an optionally substituted C2-C20 alkylene, or an optionally substituted C3-C20 heteroalkylene comprising 1-6 heteroatoms selected from P, O, N, S, and combinations thereof, wherein L can be optionally substituted with a duplex-stabilizing moiety selected from an intercalator and a minor groove binder (MGB); and
Y is an optionally substituted C2-C20 alkylene, optionally substituted C6-C10 arylene, optionally substituted C5-C10 heteroarylene, or an optionally substituted C3-C20 heteroalkylene comprising 1-6 heteroatoms selected from P, O, N, S, and combinations thereof

3. The hybridization probe of claim 1, wherein the one or more fluorinated affinity tags is attached to the 3′ end or at the 5′ end of the polynucleotide.

4. The hybridization probe of claim 1, wherein the one or more fluorinated affinity tags is attached to the one or more nucleotide units.

5. The hybridization probe of claim 1, wherein the hybridization probe comprises two, three, four, or five fluorinated affinity tags.

6. The hybridization probe of claim 1, wherein at least one fluorinated affinity tag comprises two or more polyfluorinated carbon chains.

7. The hybridization probe of claim 1, wherein (FT)ₙ-Y has two affinity tags and a structure defined by formula: 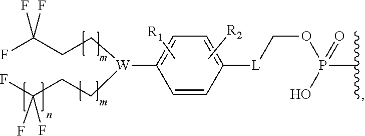
wherein:
each n, independently, is an integer from 5 to 18;
each m, independently, is 1 or 2;
W is N or a linking group comprising 1-20 carbon atoms and optionally 1-6 heteroatoms independently selected from P, O, N, and S:
R¹ and R² are independently selected from C1-C6 alkyl, halogen, nitro, amino, or cyano; or R¹ and R², together with the carbon atoms to which they are attached, can form a 5-7 membered ring optionally comprising 1-3 heteroatoms selected from P, O, N, and S; and
L is an optionally substituted C2-C20 alkylene, or an optionally substituted C3-C20 heteroalkylene comprising 1-6 heteroatoms selected from P, O, N, S, and combinations thereof.

8. The hybridization probe of claim 1, wherein (FT)ₙ-Y has two affinity tags and a structure defined by formula: 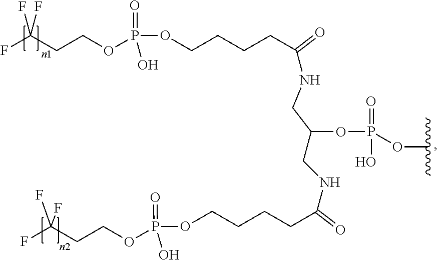
wherein n1 is an integer from 1 to 28; n2 is an integer from 1 to 28.

9. The hybridization probe of claim 1, wherein (FT)ₙ-Y has three affinity tags and a structure defined by formula: 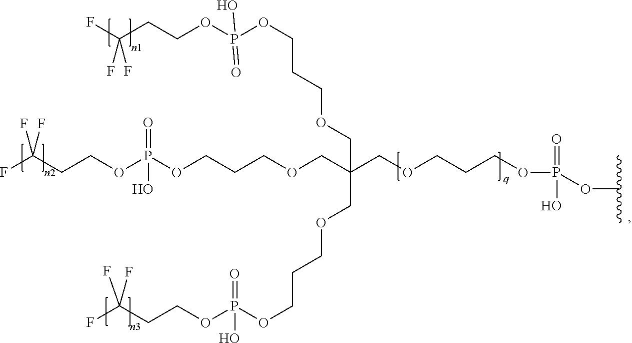
wherein n1 is an integer from 1 to 28; n2 is an integer from 1 to 28; n3 is an integer from 1 to 28; q is an integer from 0 to 10.

10. The hybridization probe of claim 1, wherein (FT)ₙ-Y has three affinity tags and a structure defined by formula:

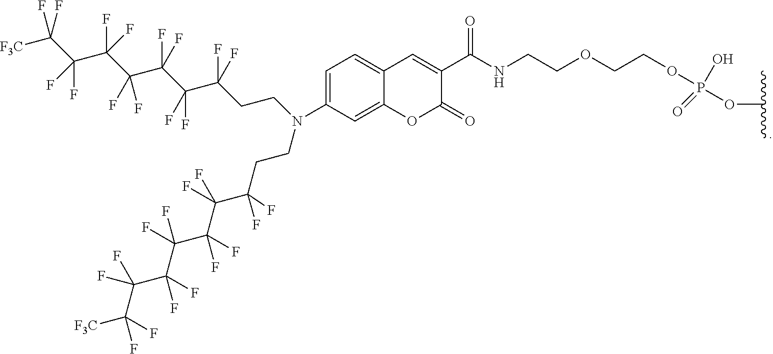 [SMILES: *OP(=O)(O)OCCOCCNC(=O)c1cc2ccc(N(CCC(F)(F)C(F)(F)C(F)(F)C(F)(F)C(F)(F)C(F)(F)C(F)(F)C(F)(F)F)CCC(F)(F)C(F)(F)C(F)(F)C(F)(F)C(F)(F)C(F)(F)C(F)(F)C(F)(F)F)cc2oc1=O]

11. The hybridization probe of claim 1, wherein (FT)ₙ-Y has three affinity tags and a structure defined by formula:

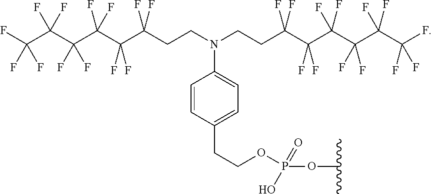 [SMILES: *OP(=O)(O)OCCc1ccc(N(CCC(F)(F)C(F)(F)C(F)(F)C(F)(F)C(F)(F)C(F)(F)F)CCC(F)(F)C(F)(F)C(F)(F)C(F)(F)C(F)(F)C(F)(F)F)cc1]

12. The hybridization probe of claim 1, having [(FT)ₙ-Y-L]ₘ- at the 5′, 3′, or any internal position of HyS.

13. The hybridization probe of claim 1, wherein the hybridization probe further comprises a stabilizing base, an intercalator, a minor groove binder, a biotin, a fluorescent dye, and/or a combination thereof.

14. The hybridization probe of claim 1, wherein the target nucleic acid is a microorganism nucleic acid or human nucleic acid.

15. A composition comprising a plurality of hybridization probes of claim 1, wherein the target nucleic acid is a microorganism nucleic acid and/or a human nucleic acid.

16. A method for enriching target nucleic acids in a mixed population of nucleic acids, wherein the mixed population of nucleic acids optionally comprises one or more target nucleic acids comprising a target sequence and one or more non-target nucleic acids, the method comprising the steps of:
a) contacting a first mixed population of nucleic acids with one or more hybridization probes of claim 1, wherein the contacting is done under conditions sufficient for formation of a duplex between the one or more hybridization probes and the target sequence, thereby providing a second mixed population of nucleic acids wherein when the mixed population comprises one or more target nucleic acids, at least a portion of the target nucleic acids comprises duplexes with the one or more hybridization probes;
b) contacting the second mixed population of nucleic acids with an affinity substrate for a time sufficient to form a complex between the fluorinated affinity tag and the affinity substrate thereby binding at least a portion of the duplexes to the affinity support;
c) separating the unbound nucleic acids from the affinity support; and
d) dissociating the duplexes between the target nucleic acids and the one or more hybridization probes bound to the affinity support, thereby generating a third mixed population of nucleic acids, wherein the ratio of the target nucleic acids to nontarget nucleic acids in the third mixed population is greater than the ratio of the target nucleic acids to non-target nucleic acids in the first mixed population.

17. The method of claim 15, wherein the one or more target nucleic acids comprises viral nucleic acids, fungal nucleic acids, bacterial nucleic acids, parasite nucleic acids, drug resistance and/or pathogenicity markers, select host nucleic acids, parasitic nucleic acids, or nucleic acids from one or more anti-microbial resistance allele regions and/or combinations thereof.

18. The method of claim 15, wherein the one or more target nucleic acids comprises human, animal, or plant nucleic acids.

19. A method for enriching nucleic acids in a mixed population of nucleic acids, the method comprising the steps of:
a) contacting a first mixed population of nucleic acids with one or more first hybridization probes and one or more second hybridization probes, wherein the first mixed population of nucleic acids comprises one or more first target nucleic acids comprising a first target sequence and one or more second target nucleic acids comprising a second target sequence, wherein the one or more first hybridization probes comprises a first affinity tag and sequence complementary to the first target sequence, and wherein the one or more second hybridization probes comprises a second affinity tag and a sequence complementary to the second target sequence, and wherein the contacting is done under conditions sufficient for formation of a duplex between the one or more first hybridization probes and the first target sequence and/or between the one or more second hybridization probes and the second target sequence;
b) contacting the mixed population of nucleic acids of step a) with a second affinity support having affinity to the second affinity tag under the conditions sufficient for formation of a complex between the second affinity tag and the second affinity support, thereby binding at least a portion of the second nucleic acids to the second affinity support;
c) separating the unbound nucleic acids of step b) from the second affinity support;
d) contacting the unbound nucleic acids of step c) with a first affinity support having affinity to the first affinity tag under the conditions sufficient for formation of a complex between the first affinity tag and the first affinity support, thereby binding at least a portion of the first nucleic acids to the first affinity support;
e) separating the unbound nucleic acids of step d) from the first affinity support; and
f) dissociating the duplexes between the first target nucleic acids and the one or more first hybridization probes, thereby generating a second mixed population of nucleic acids, wherein the ratio of the first target nucleic acids to the second nucleic acids in the second mixed population is greater than the ratio of the first target nucleic acids to the second nucleic acids in the first mixed population.

20. The method of claim 18, wherein the one or more first target nucleic acids comprise viral nucleic acids, fungal nucleic acids, bacterial nucleic acids, parasite nucleic acids, drug resistance and/or pathogenicity markers, select host nucleic acids, parasitic nucleic acids, or nucleic acids from one or more anti-microbial resistance allele regions and/or combinations thereof.

21. The method of claim 18, wherein the first hybridization probes are probes of claim 1, the first affinity support is polyfluorinated polymer, the second affinity tag is biotin, and the second affinity support comprises avidin or streptavidin.

22. The method of claim 18, wherein the second hybridization probes are probes of claim 1, the second affinity support is polyfluorinated polymer, the first affinity tag is biotin, and the first affinity support comprises avidin or streptavidin.


SEQUENCE LISTING (29 sequences)

SEQ ID NO: 1 - DNA, 80 bp, synthetic construct
  source 1..80: mol_type = other DNA
ttcatcccgtcaacattcaaacggcctgtctcatcatggaaggcgctgaatttacggaaaacattattaatggcgtcgag

SEQ ID NO: 2 - DNA, 80 bp, synthetic construct
  source 1..80: mol_type = other DNA
gtcgtggccttgctattgactctactgtagacatttttactttttatgtccctcatcgtcacgtttatggtgaacagtgg

SEQ ID NO: 3 - DNA, 80 bp, synthetic construct
  source 1..80: mol_type = other DNA
cctgaccgtaccgaggctaaccctaatgagcttaatcaagatgatgctcgttatggtttccgttgctgccatctcaaaaa

SEQ ID NO: 4 - DNA, 39 bp, synthetic construct
  source 1..39: mol_type = other DNA
attattatatattatatataaatataattaattatttta

SEQ ID NO: 5 - DNA, 80 bp, synthetic construct
  source 1..80: mol_type = other DNA
gttctaccgtcctgcactcctgtgataatccattatgttgtaaccctgaacacctatccataggaactccaaaagagaac

SEQ ID NO: 6 - DNA, 80 bp, synthetic construct; identical to SEQ ID NO: 1
  source 1..80: mol_type = other DNA

SEQ ID NO: 7 - DNA, 80 bp, synthetic construct; identical to SEQ ID NO: 2
  source 1..80: mol_type = other DNA

SEQ ID NO: 8 - DNA, 80 bp, synthetic construct; identical to SEQ ID NO: 3
  source 1..80: mol_type = other DNA

SEQ ID NO: 9 - DNA, 20 bp, synthetic construct
  source 1..20: mol_type = other DNA
ctcaaacggctggtcagtat

SEQ ID NO: 10 - DNA, 24 bp, synthetic construct
  source 1..24: mol_type = other DNA
actcgcaaggttagtgctgaggtt

SEQ ID NO: 11 - DNA, 21 bp, synthetic construct
  source 1..21: mol_type = other DNA
ctgattctgcgtttgctgatg

SEQ ID NO: 12 - DNA, 23 bp, synthetic construct
  source 1..23: mol_type = other DNA
tttagagaacgagaagacggtta

SEQ ID NO: 13 - DNA, 22 bp, synthetic construct
  source 1..22: mol_type = other DNA
tggctgctgaacgccctcttaa

SEQ ID NO: 14 - DNA, 26 bp, synthetic construct
  source 1..26: mol_type = other DNA
gggtaattatactcatcgcgaatatc

SEQ ID NO: 15 - DNA, 22 bp, synthetic construct
  source 1..22: mol_type = other DNA
gctgccgtcattgcttattatg

SEQ ID NO: 16 - DNA, 24 bp, synthetic construct
  source 1..24: mol_type = other DNA
catcccgtcaacattcaaacggcc

SEQ ID NO: 17 - DNA, 19 bp, synthetic construct
  source 1..19: mol_type = other DNA
tccgtaaattcagcgcctt

SEQ ID NO: 18 - DNA, 20 bp, synthetic construct
  source 1..20: mol_type = other DNA
tctccgtctttctccattgc

SEQ ID NO: 19 - DNA, 26 bp, synthetic construct
  source 1..26: mol_type = other DNA
cgtggccttgctattgactctactgt

SEQ ID NO: 20 - DNA, 21 bp, synthetic construct
  source 1..21: mol_type = other DNA
cgtgacgatgagggacataaa

SEQ ID NO: 21 - DNA, 39 bp, synthetic construct
  source 1..39: mol_type = other DNA
cgtgacgatgagggacataaatttaaagcgccgtggatg

SEQ ID NO: 22 - DNA, 24 bp, synthetic construct
  source 1..24: mol_type = other DNA
tgaccgtaccgaggctaaccctaa

SEQ ID NO: 23 - DNA, 20 bp, synthetic construct
  source 1..20: mol_type = other DNA
gcagcaacggaaaccataac

SEQ ID NO: 24 - DNA, 29 bp, synthetic construct
  source 1..29: mol_type = other DNA
tcatcgcgtaatgtctaatgctccgaaag

SEQ ID NO: 25 - DNA, 28 bp, synthetic construct
  source 1..28: mol_type = other DNA
  misc_feature 9
ccctgaacacctatccataggaactcca

SEQ ID NO: 26 - DNA, 49 bp, synthetic construct
  source 1..49: mol_type = other DNA
gcgacccttatttaccatgtcagtggaggtccaagctattgggtctatc

SEQ ID NO: 27 - DNA, 22 bp, synthetic construct
  source 1..22: mol_type = other DNA
ggtccaagctattgggtctatc

SEQ ID NO: 28 - DNA, 24 bp, synthetic construct
  source 1..24: mol_type = other DNA
  misc_feature 9
agcggctatcggagagagtatgct

SEQ ID NO: 29 - DNA, 22 bp, synthetic construct
  source 1..22: mol_type = other DNA
gtccttctgtgactcgcttaat